(19) United States
(12) Patent Application Publication
(10) Pub. No.: US 2022/0192897 A1
(43) Pub. Date: Jun. 23, 2022

(54) NONWOVEN WEBS WITH VISUALLY DISCERNIBLE PATTERNS AND PATTERNED SURFACTANTS

(71) Applicant: The Procter & Gamble Company, Cincinnati, OH (US)

(72) Inventors: Nathan Ray WHITELY, Liberty Township, OH (US); Kelyn Anne ARORA, Cincinnati, OH (US)

(21) Appl. No.: 17/551,234

(22) Filed: Dec. 15, 2021

Related U.S. Application Data
(60) Provisional application No. 63/127,480, filed on Dec. 18, 2020.

(51) Int. Cl. A61F 13/49; A61F 13/496

(52) U.S. Cl. CPC A61F 13/49011; A61F 13/496

(57) ABSTRACT

An absorbent article comprises a nonwoven topsheet. The nonwoven topsheet comprises a visually discernible pattern of three-dimensional features on a surface of the topsheet. The three-dimensional features comprise one or more first regions and a plurality of second regions. The one or more first regions have a first value of an average intensive property. The plurality of second regions have a second value of the average intensive property. The first value is greater than the second value and both values are greater than zero. The first regions are continuous and the second regions are discrete. A patterned surfactant is positioned on a garment-facing surface of the nonwoven topsheet. The patterned surfactant comprises a plurality of discrete, spaced apart elements. The discrete elements have an area between about 0.75 mm² and 30 mm².

### CROSS REFERENCE TO RELATED APPLICATION

This application claims the benefit, under 35 U.S.C. § 119(e), of U.S. Provisional Patent Application No. 63/127,480, filed on Dec. 18, 2020, the entire disclosure of which is incorporated herein by reference.

### FIELD

The present disclosure is generally directed to nonwoven webs with visually discernible patterns and patterned. surfactants. The present disclosure is also directed to absorbent articles comprising nonwoven webs or nonwoven topsheets with visually discernible patterns and patterned surfactants.

### BACKGROUND

Nonwoven webs are used in many industries, including the medical, hygiene, and cleaning industries. Absorbent articles comprising nonwoven webs are used in the hygiene industry to contain and absorb bodily exudates (i.e., urine, bowel movements, and menses) in infants, toddlers, children, and adults. Absorbent articles may include, but not be limited to, diapers, pants, adult incontinence products, feminine care products, and absorbent pads. Various components, such as topsheets, of these absorbent articles comprise one or more nonwoven webs. The topsheet of an absorbent article may be a rate limiting component for fluid acquisition. To drive the speed of fluid acquisition to faster times, the topsheet may be made more hydrophilic. However, this results in a topsheet that retains fluid and/or that allows fluid to traverse the topsheet from an absorbent core when under pressure, such as pressure from a wearer. Higher permeability cores may help to minimize this trade-off, but a limit may be reached when faster acquisition speeds come at the expense of wetter products. As such, nonwoven webs and nonwoven webs used as topsheets should be improved.

### SUMMARY

The present disclosure provides, in part, nonwoven webs with visually discernable patterns of three-dimensional features and with patterned surfactants. The present disclosure also provides, in part, absorbent articles comprising nonwoven webs or topsheets with visually discernable patterns of three-dimensional features and with patterned surfactants. A pattern of the visually discernable patterns of three-dimensional features may be different that a pattern of the patterned surfactant. The pattern surfactants may be applied to a garment-facing side of the topsheet to create portions of the topsheet that are hydrophilic where fluid can pass through the topsheet. The patterned surfactants may be discontinuously applied or applied in discrete zones or areas compared to topsheets with surfactants that are uniformly or continuously applied. When surfactant is uniformly or continuously applied, a trade-off exists between the speed of acquisition and the dryness of the article (fast and wet, or slow and dry). Providing the patterned surfactants in a discontinuous manner or in discrete zones or areas breaks the trade-off of fast and wet or slow and dry, especially in combination with nonwoven webs or topsheets comprising visually discernible patterns of three-dimensional features. Additional benefits of patterned surfactants include significant improvement in stain masking and potential for less bodily fluid on a wearer's skin.

The present disclosure provides, in part, an absorbent article comprising a nonwoven topsheet, a liquid impermeable backsheet, and an absorbent core positioned at least partially intermediate the topsheet and the backsheet. The nonwoven topsheet comprises a first surface, a second surface, and a visually discernible pattern of three-dimensional features on the first surface or the second surface. The three-dimensional features comprise one or more first regions and a plurality of second regions. The one or more first regions have a first value of an average intensive property. The plurality second regions have a second value of the average intensive property. The first value is greater than the second value. The first value and the second value are greater than zero. The first regions are continuous. The second regions are discrete. At least some of the first regions surround at least some of the second regions. A patterned surfactant on a garment-facing surface of the nonwoven topsheet. The patterned surfactant comprises a plurality of discrete, spaced apart elements. The discrete, spaced apart elements have an area between about 0.75 mm² and 30 mm² or between about 0.75 mm² to about 15 mm². The patterned surfactant may be hydrophilic with the remainder of the nonwoven topsheet being hydrophobic to induce absorption where the patterned surfactant is located. In other instances, the entire nonwoven topsheet may be hydrophilic, but the patterned surfactant may be more hydrophilic to induce absorption where the patterned surfactant is located. In another example, a hydrophobic composition may be applied topically in a pattern onto a nonwoven web or topsheet that is hydrophilic. The hydrophobic composition may be a lotion or topically applied triglycerides, for example. The nonwoven web or topsheet may be hydrophilic via topical or melt additive surfactants or could be naturally hydrophilic.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4;

FIG. 6 is a plan view of the absorbent article of FIG. 4, laid flat, with a garment-facing surface facing the viewer;

FIG. 7 is a cross-sectional view of the absorbent article taken about line 7-7 of FIG. 6;

FIG. 8 is a cross-sectional view of the absorbent article taken about line 8-8 of FIG. 6;

FIG. 9 is a plan view of an example absorbent core or an absorbent article;

FIG. 10 is a cross-sectional view, taken about line 10-10, of the absorbent core of FIG. 9;

FIG. 11 is a cross-sectional view, taken about line 11-11, of the absorbent core of FIG. 10;

FIG. 12 is a plan view of an example absorbent article of the present disclosure that is a sanitary napkin;

FIG. 13A is a schematic drawing illustrating a cross-section of a filament made with a primary component A and a secondary component B in a side-by-side arrangement;

FIG. 13B is a schematic drawing illustrating a cross-section of a filament made with a primary component A and a secondary component B in an eccentric sheath/core arrangement;

FIG. 13C is a schematic drawing illustrating a cross-section of a filament made with a primary component A and a secondary component B in a concentric sheath/core arrangement;

FIG. 14 is a perspective view photograph of a tri-lobal, hicomponent fiber;

FIG. 15 is a schematic representation of an example apparatus for making the nonwoven webs of the present disclosure;

FIG. 16 is a detail of a portion of the apparatus of 15 for bonding a portion of the nonwoven webs of the present disclosure;

FIG. 17 is a further detail of a portion of the apparatus for bonding a portion of the nonwoven webs of the present disclosure, taken from detail FIG. 17 in FIG. 16;

FIG. 18 is a detail of a portion of the apparatus for optional additional bonding of a portion of the nonwoven webs of the present disclosure;

FIG. 19 is a photograph of an example nonwoven web with a different design than the nonwoven webs of the present disclosure;

FIG. 20 is a photograph of a portion of a forming belt with the different design for forming nonwoven webs;

FIG. 21 is a cross-sectional depiction of a portion of the forming belt, taken about line 21-21 of FIG. 20;

FIG. 22 is an image of a portion of a mask utilized to at least in part create the forming belt of FIG. 20;

FIG. 23 is a schematic illustration of an example nonwoven web or nonwoven topsheet having a plurality of barriers and more than one visually discernible pattern of three-dimensional features for use with the absorbent articles of the present disclosure;

FIG. 24 is an example of a visually discernible pattern of three-dimensional features on a nonwoven web or a nonwoven topsheet of the present disclosure;

FIGS. 25-32 are examples of patterned surfactants for use with the nonwoven webs or nonwoven topsheets of the present disclosure;

FIG. 33 is an example of a continuous surfactant overlapped by a patterned surfactant for use with the nonwoven webs or nonwoven topsheets of the present disclosure;

FIG. 34 is a schematic cross-sectional view of a nonwoven web or nonwoven topsheet having a visually discernible pattern of three-dimensional features and with a non-registered patterned surfactant applied to a surface thereof;

FIG. 35 is a schematic cross-sectional view of nonwoven web or nonwoven topsheet having a visually discernible pattern of three-dimensional features and with a registered patterned surfactant applied to a surface thereof;

FIG. 36 is a plan view photograph of a nonwoven topsheet, and a visible stain, with a continuous surfactant applied to a garment-facing side thereof; and

FIG. 37 is a plan view photograph of a nonwoven topsheet, and a visible stain, with a patterned surfactant applied to a garment-facing side thereof.

### DETAILED DESCRIPTION

Various non-limiting forms of the present disclosure will now be described to provide an overall understanding of the principles of the structure, function, manufacture, and use of the nonwoven webs or nonwoven topsheets with visually discernable patterns and patterned surfactants disclosed herein. One or more examples of these non-limiting forms are illustrated in the accompanying drawings. Those of ordinary skill in the art will understand that the nonwoven webs or nonwoven topsheets with visually discernable patterns and patterned surfactants described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

Prior to a discussion of the nonwoven webs or nonwoven topsheets with visually discernable patterns and patterned surfactants, absorbent articles and their components and features will be discussed as one possible use of the nonwoven webs or nonwoven topsheets. It will be understood that the nonwoven webs with visually discernable patterns, sometimes with the patterned surfactants, also have other uses in other products, such as in the medical field, the cleaning and/or dusting field, and/or the wipes field, for example.

### General Description of an Absorbent Article

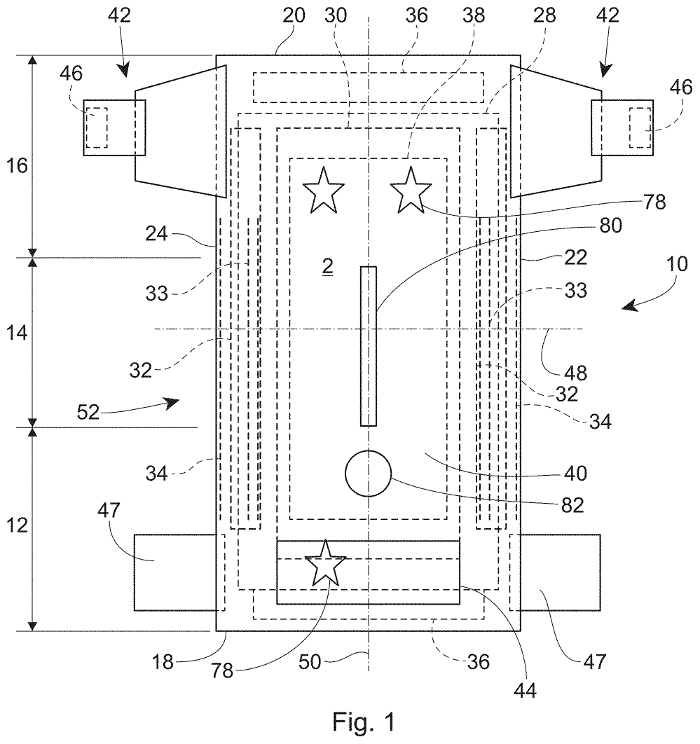

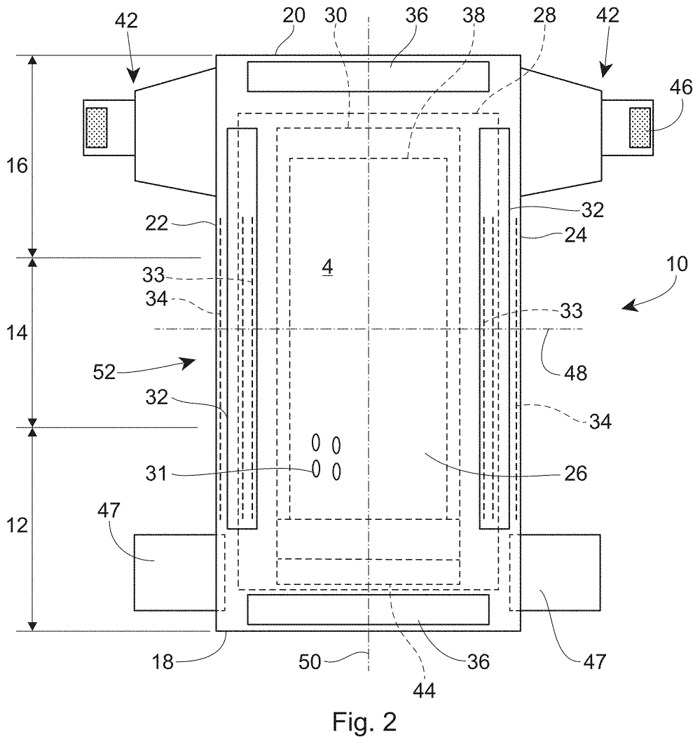

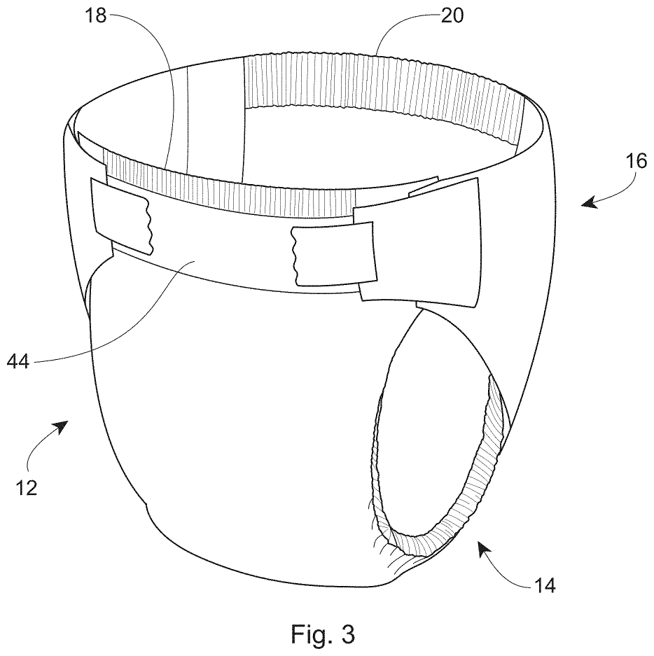

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. An outer cover nonwoven material 40, such as a nonwoven web, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. Instead of two front ears 47, the absorbent article 10 may have a single piece front belt that may function as a landing zone as well. The absorbent article 10 may have a central lateral (or transverse) axis 48 and a central longitudinal axis 50. The central lateral axis 48 extends perpendicular to the central longitudinal axis 50.

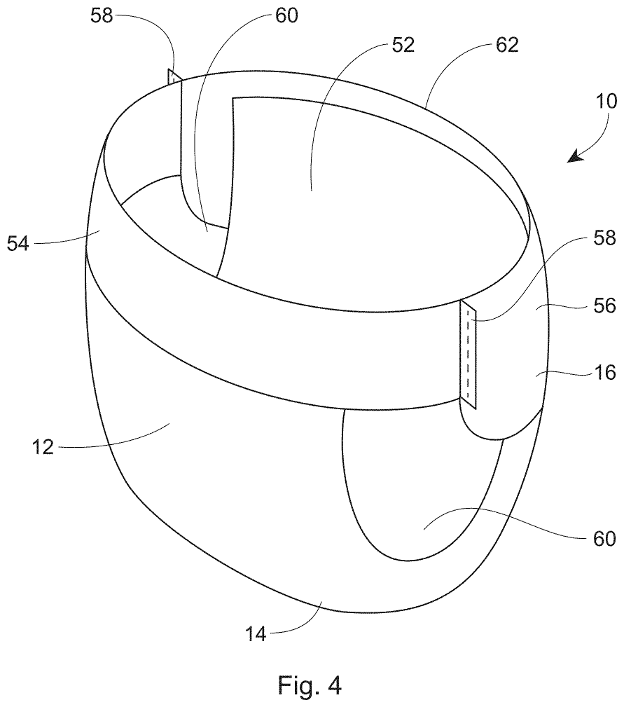

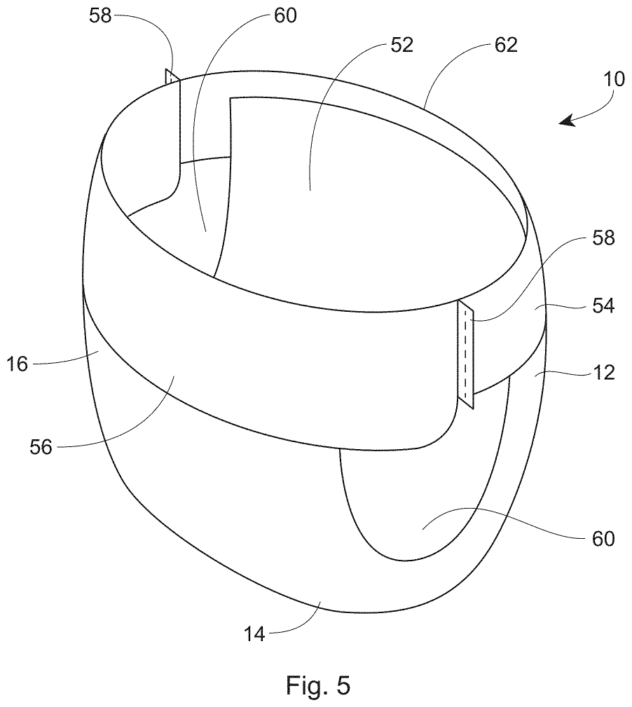

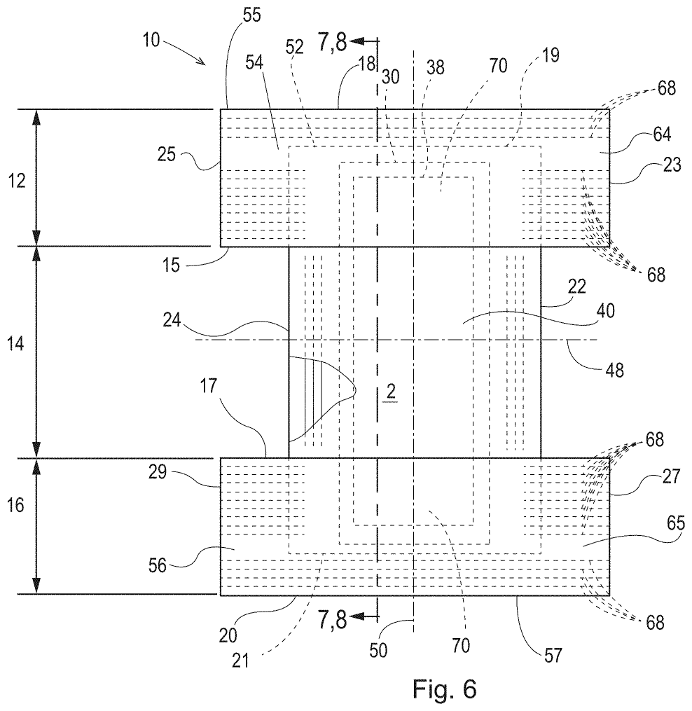

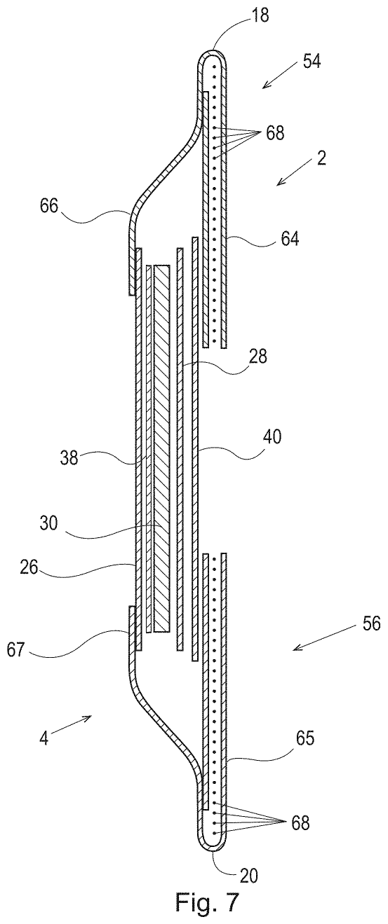

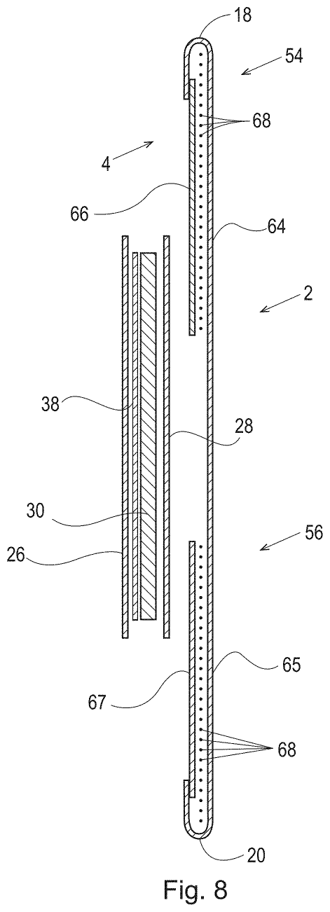

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137. Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56.

The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. Appl. Pub. No. 2013/0211363. Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt seams 15 and 17; or, alternatively, adjacent to the seams 58, 15, and 17 in the manner described in U.S. Pat. No. 9,498, 389 to create a more underwear-like article. The graphics may also be discontinuous. Alternatively, instead of attaching belts 54 and 56 to the chassis 52 to form a pant, discrete side panels may be attached to side edges of the chassis 22 and 24.

### Topsheet

The nonwoven topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as nonwoven webs, nonwoven webs of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 2, element 31), may have any suitable three-dimensional features, and/or may have a plurality of embossments (e.g., a bond pattern). Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

The nonwoven webs with visually discernable patterns and patterned surfactants disclosed herein may be used as nonwoven topsheets, or portions thereof.

### Backsheet

The backsheet 28 is generally that portion of the absorbent article 10 positioned proximate to the garment-facing surface of the absorbent core 30. The backsheet 28 may be joined to portions of the topsheet 26, the outer cover nonwoven material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet.

### Outer Cover Nonwoven Material

The outer cover nonwoven material (sometimes referred to as a backsheet nonwoven) 40 may comprise one or more nonwoven materials joined to the backsheet 28 and that covers the backsheet 28. The outer cover nonwoven material 40 forms at least a portion of the garment-facing surface 2 of the absorbent article 10 and effectively “covers” the backsheet 28 so that film is not present on the garment-facing surface 2.

### Absorbent Core

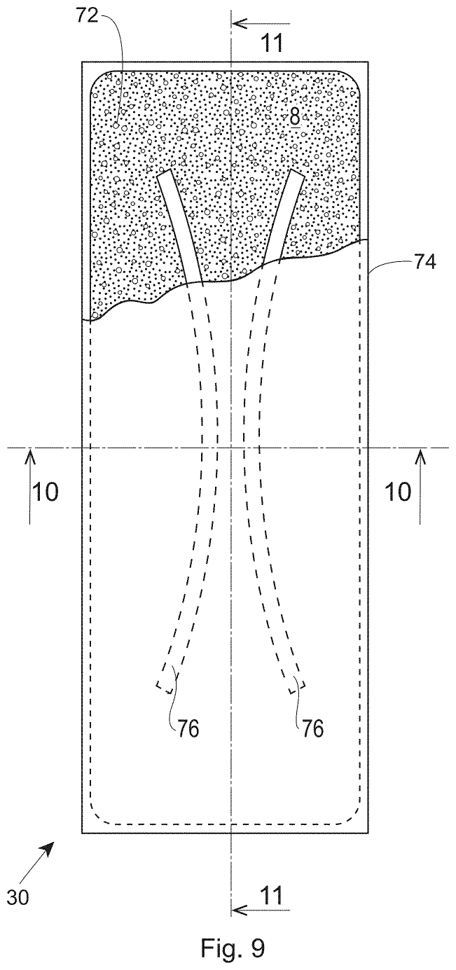

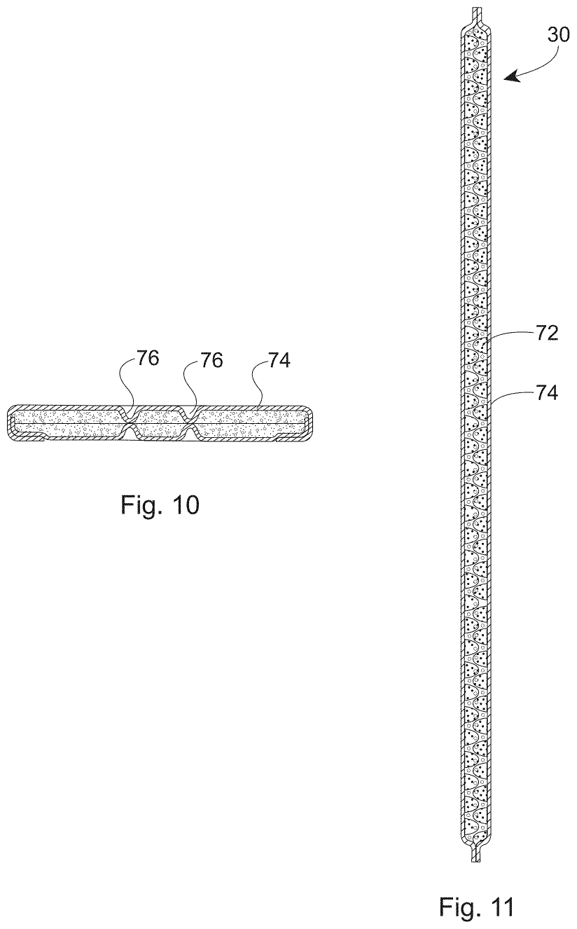

As used herein, the term “absorbent core” 30 refers to the component of the absorbent article 10 having the most absorbent capacity and that comprises an absorbent material. Referring to FIGS. 9-11, in some instances, absorbent material 72 may be positioned within a core bag or a core wrap 74. The absorbent material may be profiled or not profiled, depending on the specific absorbent article. The absorbent core 30 may comprise, consist essentially of, or consist of, a core wrap, absorbent material 72, and glue enclosed within the core wrap. The absorbent material may comprise superabsorbent polymers, a mixture of superabsorbent polymers and air felt, only air felt, and/or a high internal phase emulsion foam. In some instances, the absorbent material may comprise at least 80%, at least 85%, at least 90%, at least 95%, at least 99%, or up to 100% superabsorbent polymers, by weight of the absorbent material. In such instances, the absorbent material may be free of air felt, or at least mostly free of air felt. The absorbent core periphery, which may be the periphery of the core wrap, may define any suitable shape, such as rectangular “T,” “Y,” “hour-glass,” or “dog-bone” shaped, for example. An absorbent core periphery having a generally “dog bone” or “hour-glass” shape may taper along its width towards the crotch region 14 of the absorbent article 10.

Referring to FIGS. 9-11, the absorbent core 30 may have areas having little or no absorbent material 72, where a wearer-facing surface of the core bag 74 may be joined to a garment-facing surface of the core bag 74. These areas having little or no absorbent material and may be referred to as “channels” 76. These channels can embody any suitable shapes and any suitable number of channels may be provided. In other instances, the absorbent core may be embossed to create the impression of channels. The absorbent core in FIGS. 9-11 is merely an example absorbent core. Many other absorbent cores with or without channels are also within the scope of the present disclosure.

An absorbent core that may be used with the nonwoven topsheets described herein may comprise or be any absorbent core known in the art. The secondary topsheet/acquisition layer, intermediate the absorbent core and the topsheet, may comprise or be any secondary topsheet/acquisition layer known in the art, including spunlace and airlaid materials. These absorbent cores and/or secondary topsheets/acquisition layers may have single or multiple layers.

An absorbent core that may be used with the nonwoven topsheets described herein may have a fluid distribution layer, adjacent the topsheet and a fluid storage layer between the fluid distribution layer and the backsheet. The fluid distribution layer may be formed of two or more sub-layers, the first sub-layer proximal to the top sheet having a first amount of multiple component binder fibers or crosslinked cellulose fibers, or a combination thereof. A second and/or subsequent sub-layer distal from the topsheet comprises treated or untreated pulp and a second amount of multiple component binder fibers, crosslinked cellulose fibers, or a combination thereof. The % by weight of the first sub-layer of the first amount of multicomponent binder fibers and/or crosslinked cellulose fibers is greater than the % by weight of the second or subsequent sub-layer of the second amount of multiple component binder fibers and/or crosslinked cellulose fibers. Furthermore, the fluid storage layer has at least 50% by weight of the fluid storage layer of a super absorbent polymer.

The fluid distribution layer is configured to quickly acquire liquid from the topsheet, drawing it deep into the fluid distribution layer until such time that the liquid is absorbed by the fluid storage layer. By providing a greater % by weight of the layer of multicomponent binder fibers and/or crosslinked cellulose fibers in the first sub-layer compared with the second and/or subsequent layer provides a fluid distribution layer with a relatively more open structure in an area proximal to the topsheet. The open structure enables quick acquisition of liquid from the top sheet and has good recovery properties after liquid has been drawn down through the second sub-layer and into the fluid storage layer. The second and/or subsequent sub-layer balances the need to draw liquid from the topsheet and to retain it until absorption by the fluid storage layer, thereby preventing rewet during use of such an absorbent article.

Further details regarding the absorbent core discussed in the two above paragraphs can be found in U.S. Pat. Appl. Pub. No. 2019/0350775, filed on Mar. 15, 2019, titled Disposable Absorbent Articles. One example absorbent core is described in this reference in Table 1, inventive sample 1.

The absorbent core as contemplated herein may have any suitable x-y plane perimeter shape including but not limited to an oval shape, a stadium shape, a rectangle shape, an asymmetric shape, and an hourglass shape. In some examples, the absorbent core may be imparted with a contoured shape, e.g. narrower in an intermediate region than in the forward and rearward end regions. In other examples, the absorbent core may have a tapered shape having a wider portion in one end region of the pad which tapers to a narrower end region in the other end region of the pad. The absorbent core may stiffness that varies along one or both the longitudinal and lateral directions.

The absorbent core may have one or more layers. In certain embodiments, there are two absorbent layers where there is a first absorbent layer and a second absorbent layer adjacent to the first absorbent layer. These materials are preferably compressible, conformable, non-irritating to the wearer's skin, and capable of absorbing and retaining liquids such as urine and other certain body exudates including menses.

The first absorbent layer may include a first layer of absorbent material, which may be 100% or less of particles of superabsorbent polymer (SAP) (also known as absorbent gelling material or AGM), such as 85% to 100% SAP, 90% to 100% SAP, or even 95% to 100% SAP. The second absorbent layer may include a second layer of absorbent material, which may also be 100% or less of SAP (including the ranges specified above). Alternatively, either or both the first and second absorbent layer may include a combination of cellulose, commuted wood pulp, or the like, in combination with SAP. In some examples, the absorbent core may include a first layer and a second layer, wherein the first layer is designed primarily for absorbing and retaining fluid (sometimes known as a storage layer). The storage layer may include particles of SAP and may include particles of SAP distributed within a batt of cellulosic fiber. The second layer (sometimes known as an acquisition/distribution layer or “secondary topsheet”) may be designed to be disposed directly beneath the topsheet and configured for receiving and dispersing energy from a gush of fluid, and distributing the fluid across and down to the storage layer. The acquisition/distribution layer may be a batt or nonwoven structure of filaments or fibers which may be partially or entirely cellulosic fibers, or a blend of cellulosic fibers and polymeric fibers or filaments. In particular examples the acquisition/distribution layer may be an airlaid batt of cellulosic fibers.

Alternatively, the absorbent core may be formed entirely/solely of cellulosic fiber (including cellulosic fiber material known as “airfelt”) as the absorbent material.

The absorbent core may also comprise a carrier layer for either or both of first and second absorbent layers. This carrier layer may be a nonwoven web, which may be apertured. The absorbent core may also include a thermoplastic adhesive material at least partially bonding a layer of the absorbent material to a substrate material.

The absorbent core may include one or more grooves, channels or pockets that are defined by z-direction depressions or changes in caliper of layer(s) of the absorbent core. The one or more grooves, channels or pockets may be provided in addition to one or more channels or instead of the one or more channels in the topsheet. The pockets may be areas in the absorbent core that are free of, or substantially free of absorbent material, such as SAP (including the ranges specified above). Other forms and more details regarding channels and pockets that are free of, or substantially free of absorbent materials, such as SAP, within absorbent cores are discussed in greater detail in U.S. Pat. Appl. Pub. No. 2014/0163500; U.S. Pat. Appl. Pub. No. 2014/0163506; and U.S. Pat. Appl. Pub. No. 2014/0163511.

The configuration and construction of the absorbent core may vary (e.g., the absorbent core may have varying caliper zones, a hydrophilic gradient, a superabsorbent gradient, or lower average density and lower average basis weight acquisition zones). Further, the size and absorbent capacity of the absorbent core may also be varied to accommodate a variety of wearers. However, the total absorbent capacity of the absorbent core should be compatible with the design loading and the intended use of the sanitary napkin or any other disposable absorbent article.

In some forms contemplated herein, the absorbent core may comprise a plurality of multi-functional layers in addition to the first and second absorbent layers. For example, the absorbent core may comprise a core wrap (not shown) useful for enveloping the first and second absorbent layers and other optional layers. The core wrap may be formed by two nonwoven materials, substrates, laminates, films, or other materials. The core wrap may only comprise a single material, substrate, laminate, or other material wrapped at least partially around itself.

The absorbent core may comprise one or more adhesives, for example, to help immobilize any superabsorbent gelling material or other absorbent materials that might be present in the core.

Absorbent cores comprising relatively high amounts of SAP with various core designs are disclosed in U.S. Pat. No. 5,599,335; EP 1 447 066; WO 95/11652; U.S. Pat. Appl. Pub. No. 2008/0312622A1; and WO 2012/052172. These designs may be used to configure the first and second superabsorbent layers. Alternate core embodiments are also described in U.S. Pat. Nos. 4,610,678; 4,673,402; 4,888,231; and 4,834,735. The absorbent core may further comprise additional layers that mimic a dual core system containing an acquisition/distribution core of chemically stiffened fibers positioned over an absorbent storage core as described in U.S. Pat. No. 5,234,423 and in U.S. Pat. No. 5,147,345.

Superabsorbent polymers as contemplated herein are typically used in the form of discrete particles. Such superabsorbent polymer particles can be of any desired shape, e.g., spherical or semi-spherical, cubic, rod-like polyhedral, etc. Shapes having a large greatest dimension/smallest dimension ratio, like needles and flakes, are also contemplated for use herein. Agglomerates of fluid absorbent gelling material particles may also be used.

Some layers of an absorbent core may be substantially free of airfelt and are thus distinct from mixed layers that may include airfelt. As used herein, “substantially free of airfelt” means less than 5%, 3%, 1%, or even 0.5% of airfelt. In a preferred case, there will be no measurable airfelt in the superabsorbent layers of the absorbent core. In the case of the first superabsorbent layer, it is preferably disposed onto the first distribution layer discontinuously. As used herein “discontinuously” or “in a discontinuous pattern” means that the superabsorbent polymers are applied onto the first distribution layer in a pattern of disconnected shaped areas. These areas of superabsorbent polymers or areas free of superabsorbent polymer may include, but are not limited to linear strips, non-linear strips, circles, rectangles, triangles, waves, mesh, and combinations thereof. The first superabsorbent layer like the second superabsorbent layer may, however, be disposed onto its respective distribution layer in a continuous pattern. As used herein “continuous pattern” or “continuously” means that the material is deposited and or secured to a superabsorbent carrier material and/or the adjacent distribution layer in an uninterrupted manner such that there is rather full coverage of the distribution layer by the superabsorbent polymer.

In some examples the absorbent core may be formed of or include a layer of absorbent open-celled foam material. In some examples, the foam material may include at least first and second sublayers of absorbent open-celled foam material, the sublayers being in direct face-to-face contact with each other. In such examples, the wearer-facing sublayer may be a relatively larger-celled foam material, and the outward-facing sublayer may be a relatively smaller-celled foam material, for purposes explained in more detail below.

The open-celled foam material may be a foam material that is manufactured via polymerization of the continuous oil phase of a water-in-oil high internal phase emulsion (“HIPE”).

HIPE foams useful for forming absorbent cores and/or sublayers within contemplation of the present disclosure, and materials and methods for their manufacture, also include but are not necessarily limited to those foams and methods described in U.S. Pat. Nos. 10,045,890; 9,056,412; 8,629,192; 8,257,787; 7,393,878; 6,551,295; 6,525,106; 6,550,960; 6,406,648; 6,376,565; 6,372,953; 6,369,121; 6,365,642; 6,207,724; 6,204,298; 6,158,144; 6,107,538; 6,107,356; 6,083,211; 6,013,589; 5,899,893; 5,873,869; 5,863,958; 5,849,805; 5,827,909; 5,827,253; 5,817,704; 5,817,081; 5,795,921; 5,741,581; 5,652,194; 5,650,222; 5,632,737; 5,563,179; 5,550,167; 5,500,451; 5,387,207; 5,352,711; 5,397,316; 5,331,015; 5,292,777; 5,268,224; 5,260,345; 5,250,576; 5,149,720; 5,147,345; and U.S. Pat. Appl. Pub. No. 2005/0197414; U.S. Pat. Appl. Pub. No. 2005/0197415; U.S. Pat. Appl. Pub. No. 2011/0160326; U.S. Pat. Appl. Pub. No. 2011/0159135; U.S. Pat. Appl. Pub. No. 2011/0159206; U.S. Pat. Appl. Pub. No. 2011/0160321; U.S. Pat. Appl. Pub. No. 2011/0160689, and U.S. Pat. App. Ser. No. 62/804864.

In other examples, the absorbent core may be a heterogeneous mass formed of a nonwoven layer of spun filaments, with discrete foam pieces within and interspersed/distributed through the nonwoven structure, the discrete foam pieces being formed about and enrobing portions of filaments. Examples of such an absorbent core are described in U.S. Pat. Nos. 10,045,890; 10,016,779; 9,956,586; 9,993,836; 9,574,058; U.S. Pat. Appl. Pub. No. 2015/0313770; U.S. Pat. Appl. Pub. No. 2015/0335498; U.S. Pat. Appl. Pub. No. 2015/0374876; U.S. Pat. Appl. Pub. No. 2015/0374561; U.S. Pat. Appl. Pub. No. 2016/0175787; U.S. Pat. Appl. Pub. No. 2016/0287452; U.S. Pat. Appl. Pub. No. 2017/0071795; U.S. Pat. Appl. Pub. No. 2017/0119587; U.S. Pat. Appl. Pub. No. 2017/0119596; U.S. Pat. Appl. Pub. No. 2017/0119597; U.S. Pat. Appl. Pub. No. 2017/0119588; U.S. Pat. Appl. Pub. No. 2017/0119593; U.S. Pat. Appl. Pub. No. 2017/0119594; U.S. Pat. Appl. Pub. No. 2017/0119595; U.S. Pat. Appl. Pub. No. 2017/0199598; U.S. Pat. Appl. Pub. No. 2017/0267827; U.S. Pat. Appl. Pub. No. 2018/0110660; U.S. Pat. Appl. Pub. No. 2017/0119600; U.S. Pat. Appl. Pub. No. 2017/0119589; U.S. Pat. Appl. Pub. No. 2018/0169832; U.S. Pat. Appl. Pub. No. 2018/0168884; and U.S. Pat. Appl. Pub. No. 2018/0318150.

The absorbent core may also include similar optional layers. They may be webs selected from the group consisting of a fibrous structure, an airlaid web, a wet laid web, a high loft nonwoven, a needlepunched web, a hydroentangled web, a fiber tow, a woven web, a knitted web, a flocked web, a spunbond web, a layered spunbond/melt blown web, a carded fiber web, a coform web of cellulose fiber and melt blown filaments, a coform web of staple fibers and melt blown filaments, and layered webs that are layered combinations thereof.

These optional layers of the core and of the chassis may include materials such as creped cellulose wadding, fluffed cellulose fibers, airlaid (airfelt), and textile fibers. The materials of the optional layers may also include filaments such as, for example, synthetic fibers or filaments, thermoplastic particulates, fibers or filaments, tricomponent filaments, and bicomponent fibers or filaments such as, for example, sheath/core filaments having, for example, any of the following polymer combinations: polyethylene/polypropylene, polyethylvinyl acetate/polypropylene, polyethylene/polyester, polypropylene/polyester, copolyester/polyester, and the like. The optional layers may include any combination of the materials listed above, copolymers thereof, and/or a plurality of the materials listed above, alone or in combination.

The materials of the optional layers may be hydrophobic or hydrophilic depending on their functions and placement within or relative to the absorbent core.

The materials of the optional layers may be formed of constituent fibers or filaments including polymers such as polyethylene, polypropylene, polyester, copolymers thereof, and blends thereof. Filaments may be formed in a spunbond process. Filaments may be formed in a meltblowing process. Fibers or filaments may also be formed of or include cellulose, rayon, cotton, or other natural materials or blends of polymeric and natural materials. The fibers or filaments may also include a superabsorbent material such as polyacrylate or any combination of suitable materials. The fibers or filaments may be monocomponent, bicomponent, and/or biconstituent, non-round (e.g., capillary channel fibers), and may have major cross-sectional dimensions (e.g., diameter for round fibers) ranging from 0.1-500 microns. The constituent fibers or filaments of the nonwoven precursor web may also be a mixture of different types, differing in such features as chemistry (e.g. polyethylene and polypropylene), components (mono- and bi-), denier (micro denier and >20 denier), shape (i.e., capillary and round) and the like. The constituent fibers or filaments may range from about 0.1 denier to about 100 denier.

The optional layers may include thermoplastic particulates, fibers or filaments. The materials, and in particular thermoplastic fibers or filaments, may be made from a variety of thermoplastic polymers including polyolefins such as polyethylene and polypropylene, polyesters, copolyesters, and copolymers of any of the foregoing.

Further details regarding the absorbent core discussed above can be found in U.S. Pat. Appl. No. 16/446,052, Attorney Docket Number 15554Q, filed on Jun. 19, 2019, titled Absorbent Article with Function-Formed Topsheet, and Method for Manufacture.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14.

### Waistband

Referring to FIGS. 1 and 2, the absorbent article 10 may comprise one or more elastic waistbands 36 or non-elastic waistband. The elastic waistbands 36 may be positioned on the garment-facing surface 2 or the wearer-facing surface 4. As an example, a first elastic waistband 36 may be present in the front waist region 12 near the front belt end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven webs, foams, cellulosic materials, cross-linked cellulosic materials, air laid cellulosic nonwoven webs, spunlace materials, or combinations thereof, for example. In some instances, portions of the acquisition materials 38 may extend through portions of the topsheet 26, portions of the topsheet 26 may extend through portions of the acquisition materials 38, and/or the topsheet 26 may be nested with the acquisition materials 38. Typically, an acquisition material 38 may have a width and length that are smaller than the width and length of the topsheet 26. The acquisition material may be a secondary topsheet in the feminine pad context. The acquisition material may have one or more channels as described above with reference to the absorbent core 30 (including the embossed version). The channels in the acquisition material may align or not align with channels in the absorbent core 30. In an example, a first acquisition material may comprise a nonwoven web and as second acquisition material may comprise a cross-linked cellulosic material.

### Landing Zone

Referring to FIGS. 1 and 2, the absorbent article 10 may have a landing zone area 44 that is formed in a portion of the garment-facing surface 2 of the outer cover nonwoven material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover nonwoven material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78.

### Front and Back Ears

Referring to FIGS. 1 and 2, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. The ears, or portions thereof, may be elastic or may have elastic panels. In an example, an elastic film or elastic strands may be positioned intermediate a first nonwoven web and a second nonwoven web. The elastic film may or may not be apertured. The ears may be shaped. The ears may be integral (e.g., extension of the outer cover nonwoven material 40, the backsheet 28, and/or the topsheet 26) or may be discrete components attached to a chassis 52 of the absorbent article on a wearer-facing surface 4, on the garment-facing surface 2, or intermediate the two surfaces 4, 2.

### Sensors

Referring again to FIG. 1, the absorbent articles of the present disclosure may comprise a sensor system 82 for monitoring changes within the absorbent article 10. The sensor system 82 may be discrete from or integral with the absorbent article 10. The absorbent article 10 may comprise sensors that can sense various aspects of the absorbent article 10 associated with insults of bodily exudates such as urine and/or BM (e.g., the sensor system 82 may sense variations in temperature, humidity, presence of ammonia or urea, various vapor components of the exudates (urine and feces), changes in moisture vapor transmission through the absorbent articles garment-facing layer, changes in translucence of the garment-facing layer, and/or color changes through the garment-facing layer). Additionally, the sensor system 82 may sense components of urine, such as ammonia or urea and/or byproducts resulting from reactions of these components with the absorbent article 10. The sensor system 82 may sense byproducts that are produced when urine mixes with other components of the absorbent article 10 (e.g., adhesives, agm). The components or byproducts being sensed may be present as vapors that may pass through the garment-facing layer. It may also be desirable to place reactants in the absorbent article that change state (e.g. color, temperature) or create a measurable byproduct when mixed with urine or BM. The sensor system 82 may also sense changes in pH, pressure, odor, the presence of gas, blood, a chemical marker or a biological marker or combinations thereof. The sensor system 82 may have a component on or proximate to the absorbent article that transmits a signal to a receiver more distal from the absorbent article, such as an iPhone, for example. The receiver may output a result to communicate to the caregiver a condition of the absorbent article 10. In other instances, a receiver may not be provided, but instead the condition of the absorbent article 10 may be visually or audibly apparent from the sensor on the absorbent article.

### Packages

The absorbent articles of the present disclosure may be placed into packages. The packages may comprise nonwoven webs, polymeric films, and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate number of absorbent articles per package. By packaging the absorbent articles under compression, caregivers can easily handle and store the packages, while also providing distribution savings to manufacturers owing to the size of the packages. The nonwoven webs with visually discernable patterns and improved texture perception may be used as nonwoven components of the packages, or portions thereof.

### Sanitary Napkin

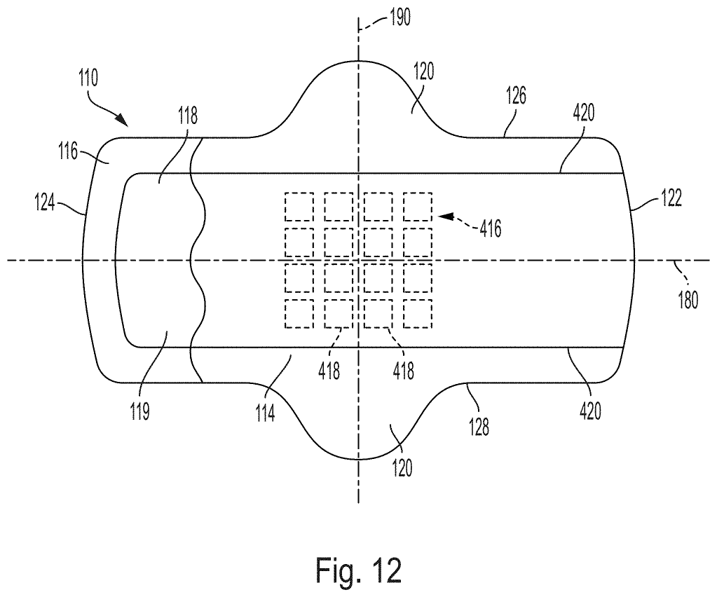

Referring to FIG. 12, an absorbent article of the present disclosure may be a sanitary napkin 110. The sanitary napkin 110 may comprise a liquid permeable topsheet 114, a liquid impermeable, or substantially liquid impermeable, backsheet 116, and an absorbent core 118. The liquid impermeable backsheet 116 may or may not be vapor permeable. The absorbent core 118 may have any or all of the features described herein with respect to the absorbent core 30 and, in some forms, may have a secondary topsheet 119 (STS) instead of the acquisition materials disclosed above. The STS 119 may comprise one or more channels, as described above (including the embossed version). In some forms, channels in the STS 119 may be aligned with channels in the absorbent core 118. The sanitary napkin 110 may also comprise wings 120 extending outwardly with respect to a longitudinal axis 180 of the sanitary napkin 110. The sanitary napkin 110 may also comprise a lateral axis 190. The wings 120 may be joined to the topsheet 114, the backsheet 116, and/or the absorbent core 118. The sanitary napkin 110 may also comprise a front edge 122, a back edge 124 longitudinally opposing the front edge 122, a first side edge 126, and a second side edge 128 longitudinally opposing the first side edge 126. The longitudinal axis 180 may extend from a midpoint of the front edge 122 to a midpoint of the back edge 124. The lateral axis 190 may extend from a midpoint of the first side edge 128 to a midpoint of the second side edge 128. The sanitary napkin 110 may also be provided with additional features commonly found in sanitary napkins as is known in the art.

The nonwoven webs or nonwoven topsheets with visually discernable patterns of three-dimensional features and patterned surfactants may be used as components of sanitary napkins, or portions thereof, such as topsheets.

Nonwoven Webs or Nonwoven Topsheets with Visually Discernible Patterns

The nonwoven webs or nonwoven topsheets with visually discernable patterns are now discussed. The nonwoven webs or nonwoven topsheets with visually discernible patterns and patterned surfactants will be discussed later. The visually discernable patterns may be formed by three-dimensional features. Such nonwoven webs may be used as various components of, or portions of components of, absorbent articles, such as topsheets, wings, outer cover nonwoven materials, belts, waistbands, leg cuffs, waist cuffs, landing zones, acquisition materials, and/or ears, for example. If the nonwoven webs are used as topsheets, the topsheets may extend into the wings of a sanitary napkin.

Any of the nonwoven webs of the present disclosure may be through-air bonded such that bonds occur at individual fiber intersections as hot air is passed through the nonwoven webs. Through-air bonding may help maintain softness in the nonwoven webs compared to more conventional calendar bonding. Other methods of bonding may include calendar point bonding, ultrasonic bonding, latex bonding, hydroentanglement, resin bonding, and/or combinations thereof.

Any of the nonwoven webs of the present disclosure may comprise portions of, or all of, components of absorbent articles. An absorbent article, as discussed above, may comprise a liquid permeable topsheet, a liquid impermeable backsheet, and an absorbent core positioned at least partially intermediate the topsheet and the backsheet. The absorbent article may comprise an outer cover nonwoven material forming at least a portion of a garment-facing surface of the absorbent article. The outer cover nonwoven material and/or the topsheet may comprise the nonwoven webs of the present disclosure. Other components of absorbent articles, or portions thereof, may also comprise the nonwoven webs of the present disclosure, such as leg cuffs, waist cuffs, belts, landing zones, waistbands, and/or ears, for example.

A nonwoven web or nonwoven topsheet for an absorbent article is provided. The nonwoven web may comprise a first surface, a second surface, and a visually discernible pattern of three-dimensional features on the first surface or the second surface. The three-dimensional features may comprise one or more first regions and a plurality of second regions. The one or more first regions are different than the plurality of second regions in a value of an average intensive property, wherein the average intensity property is basis weight, volumetric density, and/or caliper.

The nonwoven webs comprising the visually discernable patterns of three-dimensional features may have a basis weight in the range of about 10 gsm to about 100 gsm, about 10 gsm to about 60 gsm, about 15 gsm to about 50 gsm, about 15 gsm to about 45 gsm, about 20 gsm to about 40 gsm, about 20 gsm to about 35 gsm, about 20 gsm to about 30 gsm, according to the

Basis Weight Test herein.

The visually discernable pattern of three-dimensional features may be formed in a nonwoven web by embossing, hydroentangling, or by using a structured forming belt for fiber laydown. Using embossing or hydroentangling, the first regions or the second regions may be embossed or hydroentangled to form the pattern. The structured forming belt is discussed herein.

### Materials

The nonwoven webs or nonwoven topsheets of the present disclosure may be formed by a dry aid process using short staple fibers and mechanical web formation, such as a carding process. The resulting webs may be bonded using irregular pattern thermal embossing or hydroforming/hydroentangling processes. The nonwoven webs may also comprise cotton or other natural fibers. The nonwoven webs may comprise one or snore layers of meltblown fibers and/or one or more layers of spunbond fibers, Some nonwoven webs may comprises a single layer of meltblown fibers and more than one layer of spunbond fibers. Some example nonwoven webs are SMS, SMMS, SSMMS, SMMSS, SMSS, or SSMS webs. The nonwoven webs of the present disclosure may also comprise carded fibers or be solely formed of carded fibers. The nonwoven webs of the present disclosure may also be coform webs. Coformed webs typically comprise a matrix of meltblown fibers mixed with at least one additional fibrous organic materials, such as fluff pulp, cotton, and/or rayon, for example. The coform webs may be further structured by embossing or laying down the composite on a structured belt during a coforming process. In an instance, continuous spunbond filaments are used in producing the nonwoven webs if the nonwoven webs are being made on a structured forming belt (as described below). The nonwoven webs may comprise continuous mono-component polymeric filaments comprising a primary^{,} polymeric component. The nonwoven webs may comprise continuous multicomponent polymeric filaments comprising a primary polymeric component and a secondary polymeric component. The filaments may be continuous bicomponent filaments comprising a primary polymeric component A and a secondary polymeric component B. The bicomponent filaments have a cross-section, a length, and a peripheral surface. The components A and B may be arranged in substantially distinct zones across the cross-section of the bicomponent filaments and may extend continuously along the length of the bicomponent filaments. The secondary component B constitutes at least a portion of the peripheral surface of the bicomponent filaments continuously along the length of the bicomponent filaments. The polymeric components A and B may be melt spun into multicomponent fibers on conventional melt spinning equipment. The equipment may be chosen based on the desired configuration of the multicomponent. Commercially available melt spinning equipment is available from Hills, Inc. located in Melbourne, Florida. The temperature for spinning is in the range of about 180° C. to about 230° C. The bicomponent spunbond filaments may have an average diameter from about 6 microns to about 40 microns or from about 12 microns is about 40 microns, for example.

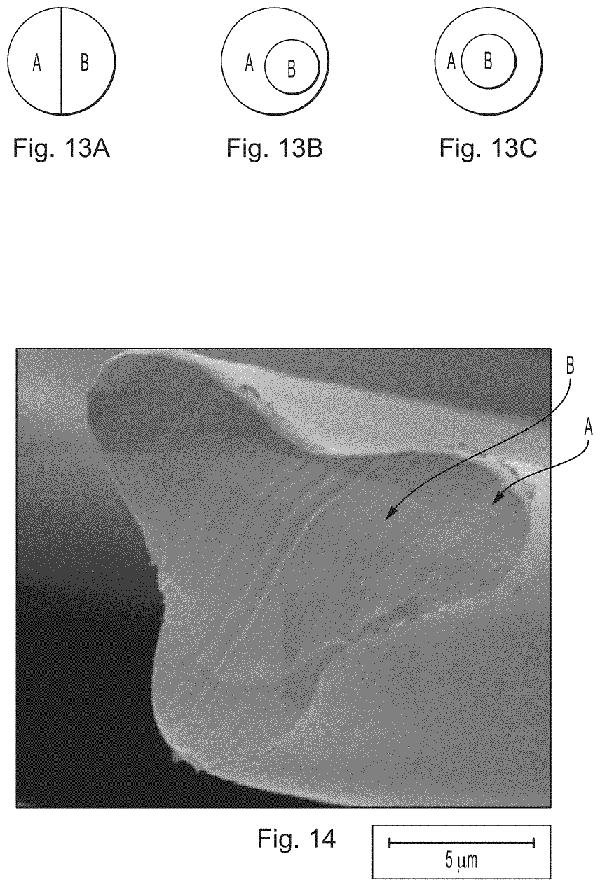

The components A and B may be arranged in either a side-by-side arrangement as shown in FIG. 13A or an eccentric sheath/core arrangement as shown in FIG. 13B to obtain filaments which exhibit a natural helical crimp. Alternatively, the components A and B may be arranged in a concentric sheath/core arrangement as shown in FIG. 13C. Additionally, the component A and B may he arranged in multi-lobal sheath/core arrangement as shown in FIG. 14. Other multicomponent fibers may be produced by using the compositions and methods of the present disclosure. The bicomponent and multicomponent fibers may be segmented pie, ribbon, islands-in-the-sea configurations, or any combination thereof. The sheath may be continuous or non-continuous around the core. The fibers of the present disclosure may have different geometries that comprise round, elliptical, star shaped, rectangular, and other various geometries. Methods for extruding multicomponent, polymeric filaments into such arrangements are generally known to those of ordinary skill in the art.

A wide variety of polymers are suitable to practice the present disclosure including polyolefins (such as polyethylene, polypropylene and polybutylene), polyesters, polyamides, polyurethanes, elastomeric materials and the like. Non-limiting examples of polymer materials that can be spun into filaments include natural polymers, such as starch, starch derivatives, cellulose and cellulose derivatives, hemicellulose, hemicelluloses derivatives, chitin. chitosan, polyisoprene (cis and trans), peptides, polyhydroxyalkanoates, and synthetic polymers including, but not limited to, thermoplastic polymers, such as polyesters, nylons, polyolefins such as polypropylene, polyethylene, polyvinyl alcohol and polyvinyl alcohol derivatives, sodium polyacrylate (absorbent gel material), and copolymers of polyolefins such as polyethylene-octene or polymers comprising monomeric blends of propylene and ethylene, and biodegradable or compostable thermoplastic polymers such as polylactic acid filaments, polyvinyl alcohol, filaments, and polycaprolactone filaments. In one example, thermoplastic polymer selected from the group of: polypropylene, polyethylene, polyester, polylactic acid, polyhydroxyalkanoate, polyvinyl alcohol, polycaprolactone, styrene-butadiene-styrene block copolymer, styrene-isoprene-styrene block copolymer, polyurethane, and mixtures thereof. In another example, the thermoplastic polymer is selected from the group consisting of: polypropylene, polyethylene, polyester, polylactic acid, polyhydroxyalkanoate, polyvinyl alcohol, polycaprolactone, and mixtures thereof. Alternatively, the polymer can comprise one derived from monomers which are bin-based such as bio-polyethylene, bio-polypropylene, bio-PET, or PLA, for example.

Primary component A and secondary component B may be selected so that the resulting bicomponent filament provides improved nonwoven bonding and softness. Primary polymer component A may have melting temperature which is lower than the melting temperature of secondary polymer component B.

Primary polymer component A may comprise polyethylene, polypropylene or random copolymer of propylene and ethylene. Secondary polymer component B may comprise polypropylene or random copolymer of propylene and ethylene. Polyethylenes may comprise linear low density polyethylene and high density polyethylene. In addition, secondary polymer component B may comprise polymers, additives for enhancing the natural helical crimp of the filaments, lowering the bonding temperature of the filaments, and enhancing the abrasion resistance, strength and softness of the resulting fabric.

Inorganic fillers, such as the oxides of magnesium, aluminum, silicon, and titanium, for example, may be added as inexpensive fillers or processing aides, Pigments and/or color melt additives may also be added.

The fibers of the nonwoven webs disclosed herein may comprise a slip additive in an amount sufficient to impart the desired haptics to the fiber. As used herein, “slip additive” or “slip agent” means an external lubricant. The slip agent when melt-blended with the resin gradually exudes or migrates to the surface during cooling or after fabrication, hence forming a uniform, invisibly thin coating, thereby yielding permanent lubricating effects. The slip agent may be a fast bloom slip agent.

During the making or in a post-treatment or even in both, the nonwoven webs of the present disclosure may be treated with surfactants or other agents to either hydrophilize the web or make it hydrophobic. For example, a nonwoven web used as a topsheet may be treated with a hydrophilizing material or surfactant so as to make it permeable to body exudates, such as urine and menses. For other absorbent articles, the nonwoven webs may remain in their naturally hydrophobic state or made even more hydrophobic through the addition of a hydrophobizing material or surfactant.

Suitable materials for preparing the multicomponent filaments of the nonwoven webs of the present disclosure may comprise PP3155 polypropylene obtained from Exxon Mobil Corporation and PP3854 polypropylene obtained from Exxon Mobil Corporation.

### Structured Forming Belts and Process for Producing Nonwoven Webs

As mentioned above, the nonwoven webs of the present disclosure may be produced by embossing, hydroentangling, or by using a structured forming belt for fiber or filament laydown. The structured forming belt and the process of manufacture will be described now in more detail than above. The nonwoven webs may be formed directly on the structured forming belt with continuous spunbond filaments in a single forming process. The nonwoven webs may assume a shape and texture which corresponds to the shape and texture of the structured forming belt.

The present disclosure may utilize the process of melt spinning. Melt spinning may occur from about 150° C. to about 280° or from about 190° to about 230°, for example. Fiber spinning speeds may greater than 100 meters/minute, from about 1,000 to about 10,000 meters/minute, from about 2,000 to about 7,000 meters/minute, or from about 2,500 to about 5,000 meters/minute, for example. Spinning speeds may affect the brittleness of the spun fiber, and, in general, the higher the spinning speed, the less brittle the fiber. Continuous fibers may be produced through spunbond methods or meltblowing processes.

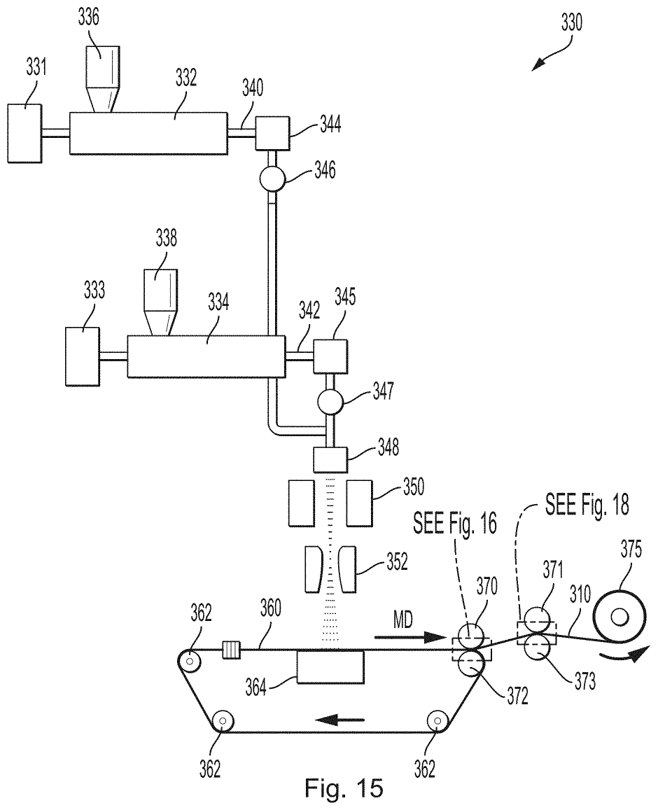

Referring to FIG. 15, a representative process line 330 for manufacturing some example nonwoven webs made on a structured forming belt of the present disclosure is illustrated. The process line 330 is arranged to produce a nonwoven web of bicomponent continuous filaments, but it should be understood that the present disclosure comprehends nonwoven webs made with monocomponent or multicomponent filaments having more than two components. The bicomponent filaments may or may not be trilobal.

The process line 330 may comprise a pair of extruders 332 and 334 driven by extruder drives 331 and 333, respectively, for separately extruding the primary polymer component A and the secondary polymer component B. Polymer component A may be fed into the respective extruder 332 from a first hopper 336 and polymer component B may be fed into the respective extruder 334 from a second hopper 338. Polymer components A and B may be fed from the extruders 332 and 334 through respective polymer conduits 340 and 342 to filters 344 and 345 and melt pumps 346 and 347, which pump the polymer into a spin pack 348. Spinnerets for extruding bicomponent filaments are generally known to those of ordinary skill in the art.

Generally described, the spin pack 348 comprises a housing which comprises a plurality of plates stacked one on top of the other with a pattern of openings arranged to create flow paths for directing polymer components A and B separately through the spinneret. The spin pack 348 has openings arranged in one or more rows. The spinneret openings form a downwardly extending curtain of filaments when the polymers are extruded through the spinneret. For the purposes of the present disclosure, spinnerets may be arranged to form side-by-side, eccentric sheath/core, or sheath/core bicomponent filaments as illustrated in FIGS. 13A-13C, as well as non-round fibers, such as tri-lobal fibers as shown in FIG. 14. Moreover, the fibers may be monocomponent having one polymeric component, such as polypropylene, for example.

The process line 330 may comprises a quench blower 350 positioned adjacent to the curtain of filaments extending from the spinneret. Air from the quench air blower 350 may quench the filaments extending from the spinneret. The quench air may be directed from one side of the filament curtain or both sides of the filament curtain.

An attenuator 352 may be positioned below the spinneret and receives the quenched filaments. Fiber draw units or aspirators for use as attenuators in melt spinning polymers are generally known to those of skill in the art. Suitable fiber draw units for use in the process of forming the nonwoven webs of the present disclosure may comprise a linear fiber attenuator of the type shown in U.S. Pat. No. 3,802,817 and eductive guns of the type shown in U.S. Pat. Nos. 3,692,618 and 3,423,266.

Generally described, the attenuator 352 may comprise an elongate vertical passage through which the filaments are drawn by aspirating air entering from the sides of the passage and flowing downwardly through the passage. A structured, endless, at least partially foraminous, forming belt 360 may be positioned below the attenuator 352 and may receive the continuous filaments from the outlet opening of the attenuator 352. The forming belt 360 may travel around guide rollers 362. A vacuum 364 positioned below the structured forming belt 360 where the filaments are deposited draws the filaments against the forming surface. Although the forming belt 360 is shown as a belt in FIG. 15, it should be understood that the forming belt may also be in other forms such as a drum. Details of particular shaped forming belts are explained below.

In operation of the process line 330, the hoppers 336 and 338 are filled with the respective polymer components A and B. Polymer components A and B are melted and extruded by the respective extruders 332 and 334 through polymer conduits 340 and 342 and the spin pack 348. Although the temperatures of the molten polymers vary depending on the polymers used, when polyethylenes are used as primary component A and secondary component B respectively, the temperatures of the polymers may range from about 190° C. to about 240° C., for example.

As the extruded filaments extend below the spinneret, a stream of air from the quench blower 350 at least partially quench the filaments, and, for certain filaments, to induce crystallization of molten filaments. The quench air may flow in a direction substantially perpendicular to the length of the filaments at a temperature of about 0° C. to about 35° C. and a velocity from about 100 to about 400 feet per minute, The filaments may be quenched sufficiently before being collected on the forming belt 360 so that the filaments may be arranged by the forced air passing through the filaments and the forming belt 360. Quenching the filaments reduces the tackiness of the filaments so that the filaments do not adhere to one another too tightly before being bonded and may be moved or arranged on the forming belt 360 during collection of the filaments on the forming belt 360 and formation of the nonwoven web.

After quenching, the filaments are drawn into the vertical passage of the attenuator 352 by a flow of the fiber draw unit. The attenuator may be positioned 30 to 60 inches below the bottom of the spinneret.

The filaments may be deposited through the outlet opening of the attenuator 352 onto the shaped, traveling forming belt 360. As the filaments are contacting the forming surface of the forming belt 360, the vacuum 364 draws the air and filaments against the forming belt 360 to form a nonwoven web of continuous filaments which assumes a shape corresponding to the shape of the structured forming surface of the structured forming belt 360. As discussed above, because the filaments are quenched, the filaments are not too tacky and the vacuum may move or arrange the filaments on the forming belt 360 as the filaments are being collected on the forming belt 330 and formed into nonwoven webs.

The process line 330 may comprise one or more bonding devices such as the cylinder-shaped compaction rolls 370 and 372, which form a nip through which the nonwoven web may be compacted (e.g., calendared) and which may be heated to bond. fibers as well, One or both of compaction rolls 370, 372 may be heated to provide enhanced properties and benefits to the nonwoven webs by bonding portions of the nonwoven webs. For example, it is believed that heating sufficient to provide thermal bonding improves the nonwoven web's tensile properties. The compaction rolls may be pair of smooth surface stainless steel rolls with independent heating controllers. The compaction rolls may be heated by electric elements or hot oil circulation. The gap between the compaction rolls may be hydraulically controlled to impose desired pressure on the nonwoven web as it passes through the compaction rolls on the forming belt. As an example, with a forming belt caliper of 1.4 mm, and a spunbond nonwoven web having a basis weight of 25 gsm, the nip gap between the compaction rolls 370 and 372 may be about 1,4 mm.

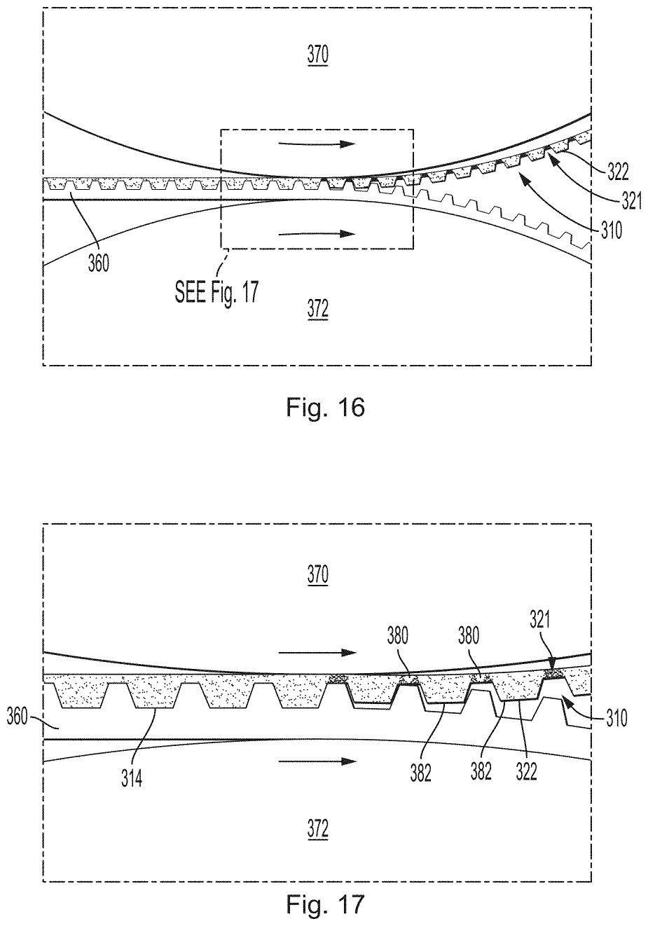

An upper compaction roll 370 may be heated sufficiently to consolidate or melt fibers on a first surface of a nonwoven web 310, to impart strength to the nonwoven web so that it may be removed from forming belt 360 without losing integrity. As shown in FIGS. 16 and 17, for example, as rolls 370 and 372 rotate in the direction indicated by the arrows, the forming belt 360 with the spunbond web laid down on it enter the nip formed by rolls 370 and 372. Heated roll 370 may heat the portions of the nonwoven web 310 that are pressed against it by the raised resin elements of belt 360, i.e., in regions 321, to create bonded fibers 380 on at least the first surface of the nonwoven web 310. As can be understood by the description herein, the bonded regions so formed may take the pattern of the raised elements of forming belt 360. By adjusting temperature and dwell time, the bonding may be limited primarily to fibers closest to the first surface of the nonwoven web 310, or thermal bonding may be achieved to a second surface. Bonding may also be a discontinuous network, for example, as point bonds 390, discussed below.

The raised elements of the forming belt 360 may be selected to establish various network characteristics of the forming belt and the bonded regions of the nonwoven web 310. The network corresponds to resin making up the raised elements of the forming belt 360 and may comprise substantially continuous, substantially semi-continuous, discontinuous, or combinations thereof options. These networks may be descriptive of the raised elements of the forming belt 360 as it pertains to their appearance or make-up in the X-Y planes of the forming belt 360 or the three-dimensional features of the nonwoven webs 310.

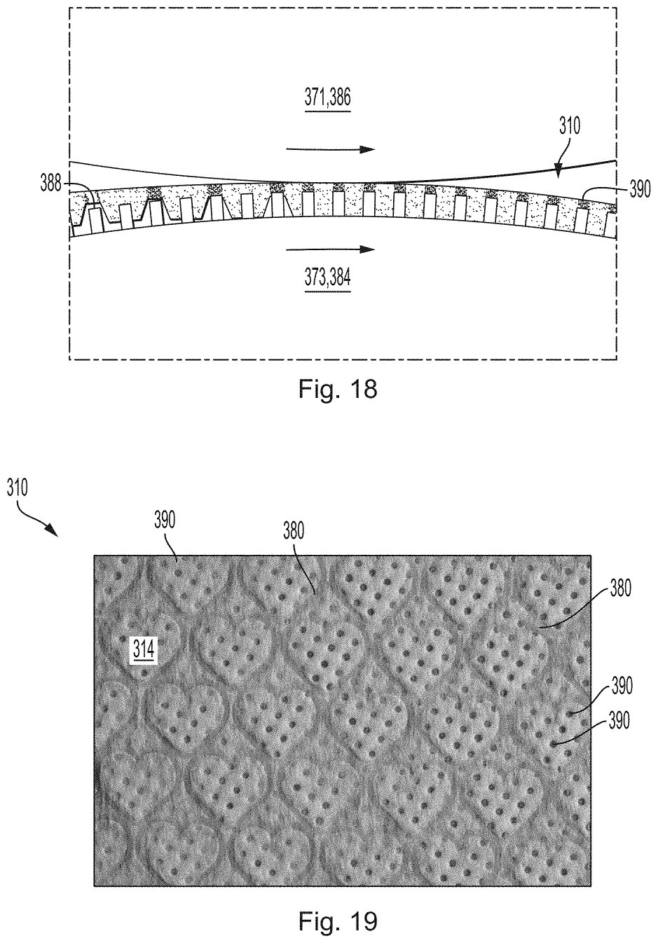

After compaction, the nonwoven web 310 may leave the forming belt 360 and be calendared through a nip formed by calendar rolls 371, 373, after which the nonwoven web 310 may be wound onto a reel 375 or conveyed directly into a manufacturing operation for products, such as absorbent articles. As shown in the schematic cross-section of FIG. 18, the calendar rolls 371, 373 may be stainless steel rolls having an engraved pattern roll 384 and a smooth roll 386. The engraved roll may have raised portions 388 that may provide for additional compaction and bonding to the nonwoven web 310. Raised portions 388 may be a regular pattern of relatively small spaced apart “pins” that form a pattern of relatively small point bonds 390 in the nip of calendar rolls 371 and 373. The percent of point bonds in the nonwoven web 10 may be from about 3% to about 30% or from about 7% to about 20%, for example. The engraved pattern may be a plurality of closely spaced, regular, generally cylindrically-shaped, generally flat-topped pin shapes, with pin heights being in a range of about 0.5 mm to about 5 mm or from about 1 mm to about 3 mm, for example. Pin bonding calendar rolls may form closely spaced, regular point bonds 390 in the nonwoven web 10, as shown in an example in FIG. 19. Further bonding may be by hot-air-through bonding, for example. FIG. 19 shows a hearts pattern made by the same structured forming belt technology that may be used to make the nonwoven webs of the present disclosure.

“Point bonding”, as used herein, is a method of thermally bonding a nonwoven web. This method comprises passing a web through a nip between two rolls comprising a heated male patterned or engraved metal roll and a smooth or patterned metal roll, The male patterned roll may have a plurality of raised, generally cylindrical-shaped pins that produce circular point bonds. The smooth roll may or may not be heated, depending on the application. In a nonwoven manufacturing line, the nonwoven web, which could be a non-bonded nonwoven web, is fed into the calendar nip and the fiber temperature is raised to the point for fibers to thermally fuse with each other at the tips of engraved points and against the smooth roll. The heating time is typically in the order of milliseconds. The nonwoven web properties are dependent on process settings such as roll temperatures, web line speeds, and nip pressures, all of which may be determined by the skilled person for the desired level of point bonding. Other types of point bonding known generally as hot calendar bonding may use different geometries for the bonds (other than circular shaped), such as oval, lines, circles, for example. In an example, the point bonding produces a pattern of point bonds being 0.5 mm diameter circles with 10% overall bonding area. Other bonding shapes may have raised pins having a longest dimension across the bonding surface of a pin of from about 0.1 mm to 2.0 mm and the overall bonding area ranges from about 5% to about 30%, for example.

As shown in FIG. 19, a heated compaction roll 370 may form a bond pattern, which may be a substantially continuous network bond pattern 380 (e.g., interconnected heart shaped bonds) on a first surface of the nonwoven web 310 (not shown in FIG. 19, as it faces away from the viewer), and the engraved calendar roll 373 may form relatively small point bonds 390 on a second surface 314 of the nonwoven web. The point bonds 390 may secure loose fibers that would otherwise be prone to fuzzing or pilling during use of the nonwoven web 310. The advantage of the resulting structure of the nonwoven web 310 is most evident when used as a topsheet or outer cover nonwoven material in an absorbent article, such as a diaper, for example. In use, in an absorbent article, a first surface of the nonwoven web 310 may be relatively flat (relative to second surface 14) and have a relatively large amount of bonding due to the heated compaction roll forming bonds 380 at the areas of the nonwoven web pressed by the raised elements of the forming belt 360. This bonding gives the nonwoven web 310 structural integrity, but still may be relatively stiff or rough to the skin of a user. Therefore, a first surface of the nonwoven web 310 may be oriented in a diaper or sanitary napkin o face the interior of the article, i.e away from the body of the wearer or garment-facing. Likewise, the second surface 314 may be wearer-facing in use, and in contact with the body. The relatively small point bonds 390 may be less likely to be perceived visually or tacitly by the user, and the relatively soft three-dimensional features may remain visually free of fuzzing and pilling while feeling soft to the body in use. Further bonding may be used instead of, or in addition to, the above-mentioned bonding. Through-air bonding may also be used.

The forming belt 360 may be made according to the methods and processes described in U.S. Pat. No. 6,610,173, issued to Lindsay et al., on Aug. 26, 2003, or U.S. Pat. No. 5,514,523, issued to Trokhan et al., on May 7, 1996, or U.S. Pat. No. 6,398,910, issued to Burazin et al., on Jun. 4, 2002, or U.S. Pat. No. 8,940,376, issued to Stage et al on Jan. 27, 2015, each with the improved features and patterns disclosed herein for making spunbond nonwoven webs. The Lindsay, Trokhan, Burazin, and Stage disclosures describe structured forming belts that are representative of paper making belts made with cured resin on a woven reinforcing member, which belts, with improvements, may be utilized to form the nonwoven webs of the present disclosure as described herein.

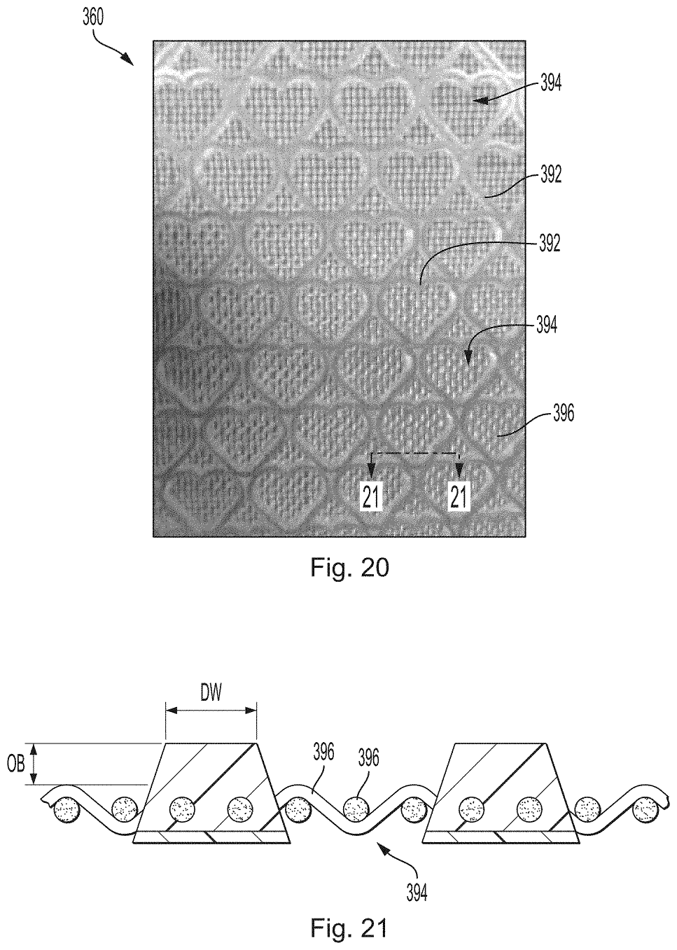

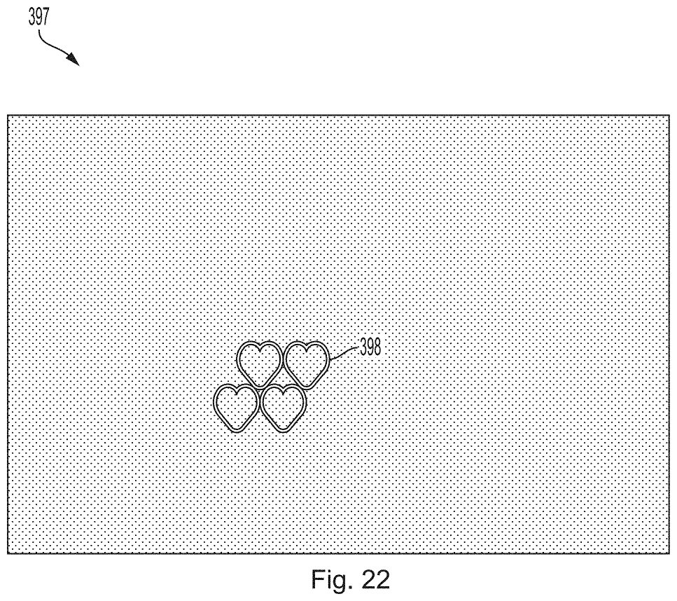

An example of a structured forming belt 360, and which may he made according to the disclosure of U.S. Pat. No. 5,514,523, is shown in FIG. 20. As taught therein, a reinforcing member 394 (such as a woven belt of filaments 396) is thoroughly coated with a liquid photosensitive polymeric resin to a preselected thickness. A film or negative mask incorporating the desired raised element pattern repeating elements (e.g., FIG. 22) is juxtaposed on the liquid photosensitive resin. The resin is then exposed to light of an appropriate wave length through the film, such as UV light for a UV-curable resin. This exposure to light causes curing of the resin in the exposed areas white portions or non-printed portions in the mask). Uncured resin (resin under the opaque portions in the mask) is removed from the system leaving behind the cured resin forming the pattern illustrated, for example, the cured resin elements 392 shown in FIG. 20.

The forming belt 360 may comprise cured resin elements 392 on a woven reinforcing member 394. The reinforcing member 394 may he made of woven filaments 396 as is generally known in the art of papermaking belts, including resin coated papermaking belts. The cured resin elements may have the general structure depicted in FIG. 20, and are made by the use of a mask 397 having the dimensions indicated in FIG. 22 As shown in schematic cross-section in FIG. 21, cured resin elements 392. flow around and are cured to “lock on” to the reinforcing member 394 and may have a width at a distal end DW of about 0.020inches to about 0.060 inches, or from about 0.025 inches to about 0.030 inches, and a total height above the reinforcing member 394, referred to as over burden, OB, of about 0.030 inches to about 0.120 inches or about 0,50 inches to about 0.80 inches, or about 0.040 inches. FIG. 22 represents a portion of a mask 397 showing the design and representative dimensions for one repeat unit of the repeating hearts design, shown herein merely as an example. The white portion 398 is transparent to UV light, and in the process of making the belt, as described in U.S. Pat. No. 5,514,523, permits UV light to cure an underlying layer of resin which is cured to form the raised elements 392 on the reinforcing member 394. After the uncured resin is washed away, the forming belt 360 having a cured resin design as shown in FIG. 20 is produced by seaming the ends of a length of the forming belt, the length of which may be determined by the design of the apparatus, as depicted in FIG. 15.

The nonwoven webs disclosed herein may be fluid permeable. The entire nonwoven web may be considered fluid permeable or some regions may be fluid permeable. By fluid permeable, as used herein, with respect to the nonwoven web is meant that the nonwoven web has at least one region which permits liquid to pass through under in-use conditions of a consumer product or absorbent article. For example, if used as a topsheet on a disposable absorbent article, the nonwoven web may have at least one zone having a level of fluid permeability permitting urine to pass through to an underlying absorbent core. By fluid permeable, as used herein with respect to a region, it is meant that the region exhibits a porous structure that permits liquid to pass through.

Because of the nature of the structured forming belts and other apparatus elements, as described herein, the three-dimensional features of the nonwoven web have average intensive properties that may differ between first and second regions, or from feature to feature in ways that provide for beneficial properties of the nonwoven web when used in personal care articles, garments, medical products, and cleaning products. For example, a first region may have a basis weight or density that is different from the basis weight or density of a second region, and both may have a basis weight or density that is different from that of a third region, providing for beneficial aesthetic and functional properties related to fluid acquisition, distribution and/or absorption in diapers or sanitary napkins.

The average intensive property differential between the various regions of the nonwoven webs is believed to be due to the fiber distribution and compaction resulting from the apparatus and method described herein. The fiber distribution occurs during the fiber laydown process, as opposed to, for example, a post making process such as embossing processes. Because the fibers are free to move during a process such as a melt, spinning process, with the movement determined by the nature of the features and air permeability of the forming belt and other processing parameters, the fibers are believed to be more stable and permanently formed in nonwoven web.

In structured forming belts having multiple zones, the air permeability in each zone may be variable such that the intensive properties of average basis weight and average volumetric density in the zones may be varied. Variable air permeabilities in the various zones causes fiber movement during laydown. The air permeability may be between about 400 to about 1000 cfm, or between about 400 to about 800 cfm, or between about 500 cfm and about 750 cfm, or between about 650 to about 700 cfm.

A structured forming belt may comprise an endless foraminous member comprising a first surface and a second surface, a curable resin extending from the first surface of the foraminous member, and a visually discernible pattern of three-dimensional features on the endless foraminous member. The three-dimensional features may comprise one or more first regions and a plurality of second regions. The one or more first regions may comprise the resin and the plurality of second regions may be free of the resin.

Nonwoven webs may comprise multicomponent fibers or bicomponent fibers, where at least one or more of the components are bio-based. Examples include side-by-side, sheath/core, or islands in the sea configurations, where one or more or all of the components are bio-based.

### Emtec

The nonwoven webs of the present disclosure provide improved softness even with the texture. The nonwoven webs of the present disclosure further solve the contradiction between high softness and high visible texture. Softness, texture smoothness), and/or stiffness may be measured by an Emtec Tissue Softness Analyzer, according to the Emtec Test herein. Tactile softness is measured as TS7. Texture/Smoothness is measured as TS750. Stiffness is measured as D.

A portion of, or all of, the nonwoven webs of the present disclosure may have a TS7 value in the range of about 1 dB V² rms to about 4.5 dB V² rms, about 2 dB V² rms to about 4.5 dB V² rms, or about 2 dB V² rms to about 4.0 dB V² rms. The portion of, or all of, the nonwoven webs of the present disclosure may also have a TS750 value in the range of about 4 dB V² rms to about 30 dB V² rms, about 6 dB V² rms to about 30 dB V² rms, about 6 dB V² rms to about 20 dB V² rms, about 6 dB V² rms to about 15 dB V² rms, about 6 dB V² rms to about 12 dB V² rms, or about 6.5 dB V² rms to about 10 dB V² rms. The portion of, or all of, the wearer-facing surfaces of the topsheets of the present disclosure may also have a D value in the range of about 1 mm/N to about 10 mm/N, about 3 mm/N to about 8 mm/N, about 2 mm/N to about 6 mm/N, about 2 mm/N to about 4 mm/N, or about 3 mm/N to about 4 mm/N. All values are measured according to the Emtec Test herein. The TS7 value is tactile softness, so low numbers are desired (the lower the number, the more soft the material is). The TS750 value is texture so a high number is desired (the higher the number, the more texture the material has). Having a low TS7 value and a high texture value is contradictory in that typically the more texture a nonwoven fabric has, the less soft it is. The Applicants, without wishing to be bound by theory, have discovered the unexpected results of highly textured nonwoven fabrics that still are very soft.

Nonwoven Webs or Nonwoven Topsheets with Improved Softness

The nonwoven webs for absorbent articles of the present disclosure result in improved softness. The nonwoven webs for absorbent articles may comprise a first surface, a second surface, and a visually discernible pattern of three-dimensional features on the first surface and/or the second surface. The nonwoven webs may comprise continuous fibers. The three-dimensional features may comprise one or more, or a plurality of, first regions and a plurality of second regions. The one or more first regions may have a first value of an average intensive property. The plurality second regions may have a second value of the average intensive property. The first value and the second value may be different and are both greater than zero.

The nonwoven webs may comprise bonds at fiber intersections formed by passing hot air through the nonwoven webs and using a process referred to as through-air bonding. In other instances, the nonwoven webs may be hydroentangled. In other instances, the nonwoven webs may comprise calendar bonds configured to join the fibers together. In still other instances, the nonwoven webs may be formed on a structured forming belt as described herein with respect to FIGS. 15-22.

The nonwoven web of the present disclosure may comprise a second, visually discernible pattern of three-dimensional features on the first surface or the second surface. The second, visually discernible pattern of three-dimensional features may be different than the visually discernible pattern. The three-dimensional features may comprise one or more, or a plurality of, third regions and a plurality of fourth regions. The one or more third regions may be different than the plurality of fourth regions in a value of an average intensive property, such as basis weight, caliper, and/or volumetric density.

The nonwoven webs of the present disclosure may comprise multicomponent fibers, such as bicomponent fibers (see e.g., FIGS. 13A-13C). At least one component of the multicomponent fibers may be bio-based, such as PLA, bio-PE, or bio-PP, for example.

The nonwoven webs of the present disclosure may have a TS7 value in the range of about 1 dB V² rms to about 4.5 dB V² rms, according to the Emtec Test, and a TS750 value in the range of about 6 dB V² rms to about 30 dB V² rms, according to the Emtec Test. The nonwoven webs of the present disclosure may have a D value in the range of about 2 mm/N to about 6 mm/N, according to the Emtec Test. The ranges of TS7, TS750, and D characterize the improved softness of the nonwoven webs or nonwoven topsheets of the present disclosure.

The nonwoven webs discussed herein may form at least portions of, or all of, one or nonwoven components of absorbent articles, such as the nonwoven components discussed above. In some instances, the nonwoven webs may form topsheets of absorbent articles.

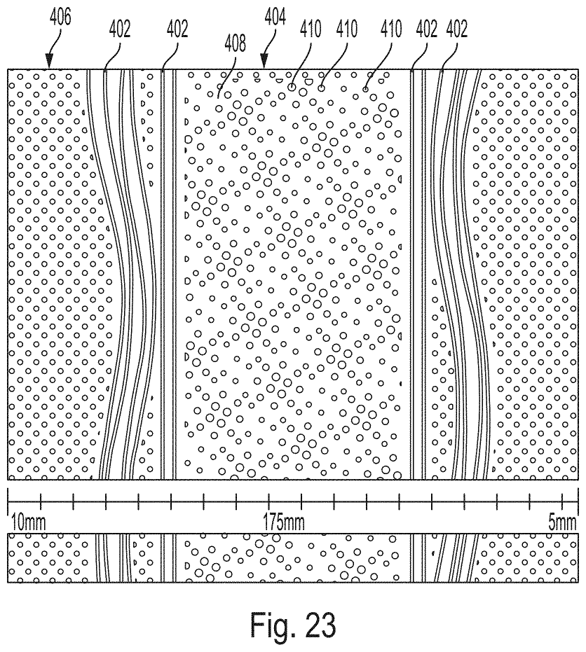

FIG. 23 is a schematic illustration of an example nonwoven web or nonwoven topsheet 400 having a plurality of longitudinally extending barriers 402, a first visually discernible pattern of three-dimensional features 404, and a second visually discernible pattern of three-dimensional features 406, on a first surface or a second surface of the nonwoven web or topsheet, for use with the absorbent articles of the present disclosure. The longitudinally extending barriers 402 may be linear and may be continuous or discontinuous. The longitudinally extending barrier 402 may form straight lines or wavy lines. The longitudinally extending barriers 402 may form a third visually discernible pattern of three-dimensional features. The white portions in FIG. 23 indicate second regions 410 and the black portions indicate first regions 408. In a zone between the longitudinally extending barriers 402, the second regions 410 may be discrete or discontinuous and the first regions 408 may be continuous. In a zone comprising the longitudinally extending barriers 402, the second regions 410 may be continuous and the first regions 408 may be continuous. In the zones outboard of the barriers 402, the second regions 410 may be discrete and the first regions 408 may be continuous. p The three-dimensional features in both the first and second visually discernible patterns of three-dimensional features 404 and 406 may have one or more or a plurality of first regions 408 and a plurality of second regions 410. The one or more first regions 408 may be different than the plurality of second regions 410 in a value of an average intensive property (i.e., caliper, volumetric density, and/or basis weight). The first and second visually discernible patterns of three-dimensional features 404 and 406 may be free of overlap with each other.

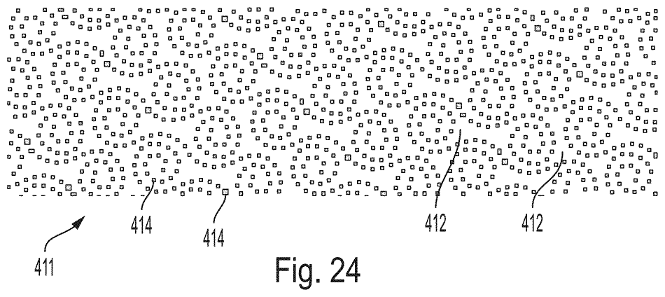

FIG. 24 is an example of a visually discernible pattern of three-dimensional features on a first or second surface of a nonwoven web or a nonwoven topsheet 411 of the present disclosure. The visually discernible pattern of three-dimensional features may comprise one or more first regions 412 and a plurality of second regions 414. The one or more first regions 412 may have a first value of an average intensive property. The plurality of second regions 414 may have a second value of an average intensive property. The first value may be greater than, less than, or different than, the second value. Both the first value and the second value may be greater than zero. The intensive property may be basis weight, volumetric density, or caliper. The one or more first regions 412 may be continuous. At least some of, or all of, the one or more first regions 412 may surround at least some of, or all of, the plurality of second regions 414. The plurality of second regions 414 may be discrete. Other visually discernible patterns are also contemplated, some of which may have continuous second regions and discontinuous first regions. The nonwoven topsheets herein may comprise a polypropylene/polypropylene side-by-side bicomponent fibers comprising up to 1.5% by weight of erucamide or other hydrophobic melt additive by weight of the nonwoven topsheet. The fibers may have fiber diameter in the range of about 15 um to about 25 um, about 15 um to about 20 um, or about 18 um.

### Patterned Surfactant

The present disclosure provides, in part, nonwoven webs or nonwoven topsheets with visually discernable patterns of three-dimensional features and with patterned surfactants. The present disclosure also provides, in part, absorbent articles comprising nonwoven webs or nonwoven topsheets with visually discernable patterns of three-dimensional features and with patterned surfactants. The pattern surfactants may be applied to a core-facing side of the topsheet to create portions of the topsheet that are hydrophilic where fluid can pass through the topsheet. The patterned surfactants may be discontinuously applied or applied in discrete zones or areas compared to topsheets with surfactants that are uniformly applied. The patterned surfactants are typically hydrophilic with the remainder of the nonwoven topsheet being hydrophobic to induce absorption where the patterned surfactant is located. In other instances, the patterned surfactants are hydrophilic with the remainder of the nonwoven topsheets being less hydrophilic to induce absorption where the patterned surfactant is located. When surfactant is uniformly applied, a trade-off exists between the speed of acquisition and the dryness of the article (fast and wet, or slow and dry). Providing the patterned surfactants in a discontinuous manner or in discrete zones or areas breaks the trade-off of fast and wet or slow and dry, especially in combination with nonwoven webs or topsheets comprising visually discernible patterns of three-dimensional features. Additional benefits of patterned surfactants include significant improvement in stain masking and potential for less bodily fluid on a wearer's skin. The patterned surfactant may comprise any surfactant suitable for nonwoven webs. One example surfactant is Stantex S6887, supplied by Pulcra Chemicals.

Referring again to FIG. 12, a sanitary napkin 110 may have wings 120 and a nonwoven topsheet 114. The wings 120 may extend outwardly relative to the central longitudinal axis 180 of a sanitary napkin. The nonwoven topsheet 114 may extend fully or partially into the wings. The nonwoven topsheet 114 may comprise a patterned surfactant 416 on a garment-facing surface of the nonwoven topsheet. A patterned surfactant may also be applied to a garment-facing surface of a diaper, pant, or other absorbent article and is only illustrated on a sanitary napkin as an example. The patterned surfactant 416 may comprise a plurality of discrete, spaced apart elements 418. The discrete, spaced apart elements 418 may have an area in the range of about 0.75 mm² to about 30 mm², or about 0.75 mm² to about 15 mm², according to Composition Pattern Analysis Test. Portions of the nonwoven topsheet 114 not having the patterned surfactant 416 may be hydrophobic or may be less hydrophilic than areas having the patterned surfactant 416. The topsheet 114 may have a visually discernible pattern of three-dimensional features illustrated in FIG. 23 or FIG. 24 or may have another visually discernible pattern of three-dimensional features. The visually discernible pattern of three-dimensional features of a nonwoven topsheet may be different than a pattern of a patterned surfactant.

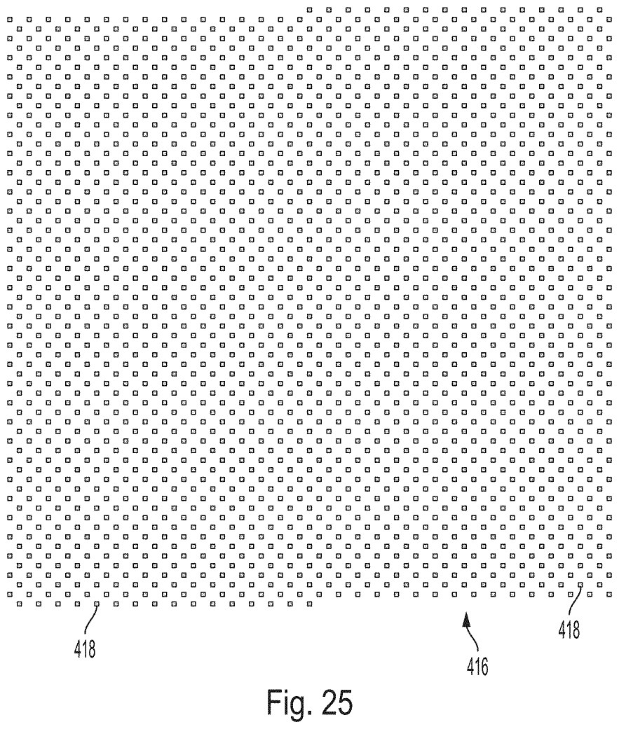

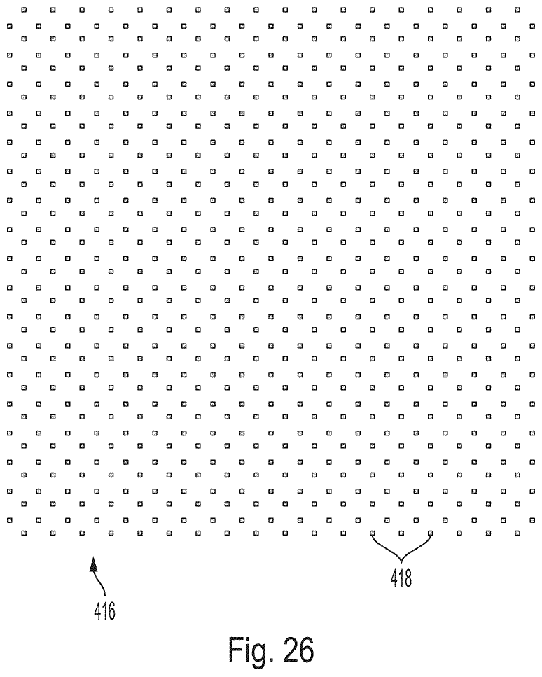

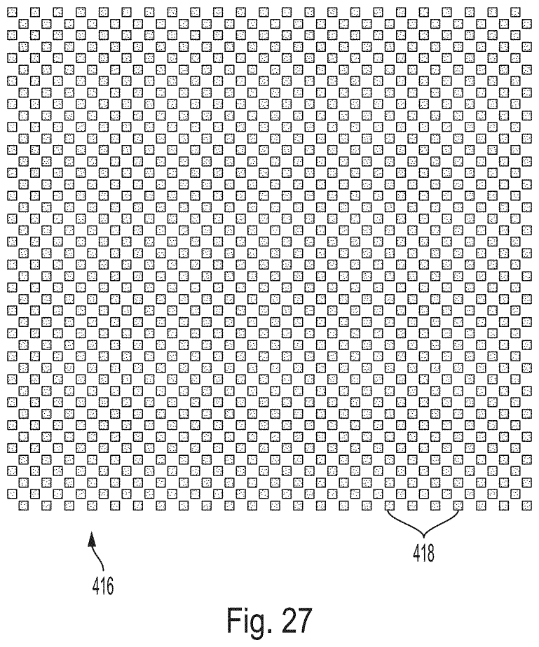

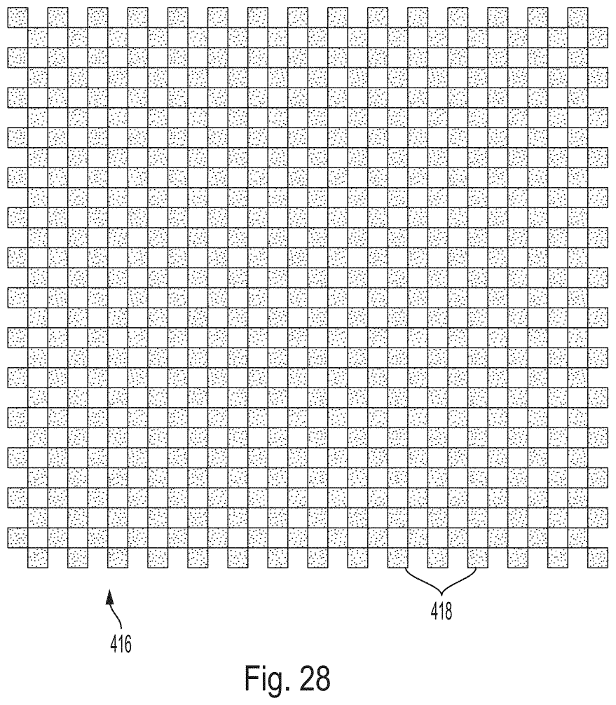

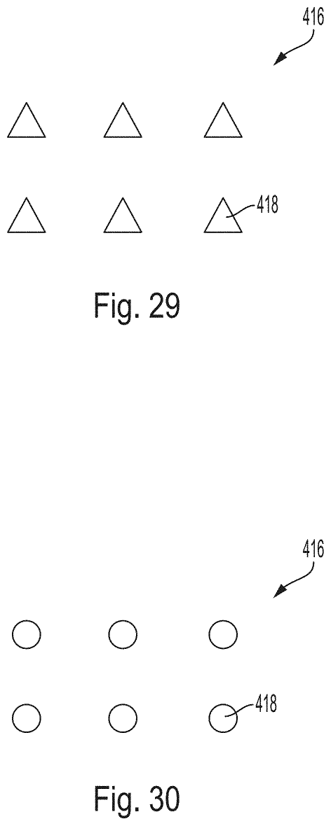

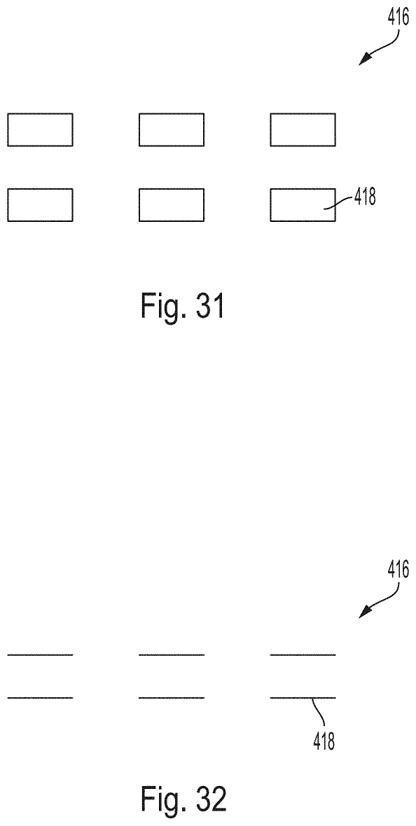

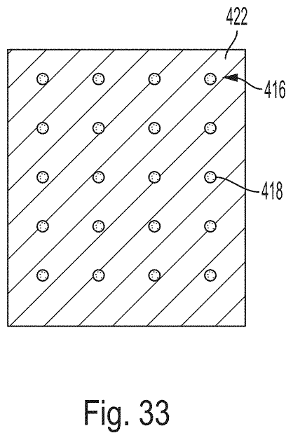

The nonwoven topsheet 114 may comprise two or more longitudinally extending barriers 420 similar to the longitudinal extending barriers 402 illustrated in FIG. 23. The patterned surfactant 416 may be positioned intermediate the longitudinally extending barriers 420. The longitudinally extending barriers 420 may be positioned inboard of the wings 120 or may cross through portions of the wings 120. The patterned surfactant 416 may only be present intermediate the longitudinally extending barriers 402 and may not be present in the wings 120. The wings 120 may be free of any surfactant, patterned or not. The patterned surfactant 416 may be concentrated in a fluid discharge location in an absorbent article, but may also be positioned at other locations. The discrete, spaced apart elements 418 of the patterned surfactant 416 may have any suitable shape, such as squares, hearts, diamonds, rectangles, triangles, circles, linear elements, ovals, pentagons, or the like. In some instances, the discrete, spaced part elements 418 may form polygonal shapes. FIGS. 25-32 are examples of patterned surfactants 416 having discrete, spaced apart elements 418 for use with the nonwoven webs or nonwoven topsheets of the present disclosure, although other patterns are also contemplated. An aspect ratio of the discrete, spaced apart elements may be about 0.5 to about 10, about 0.5 to about 5, about 0.5 to about 3, about 0.5 to about 2, about 0.5 to about 1.5, or about 1, for example. Discrete, spaced apart elements are preferred compared to continuous or high aspect ratio elements for the patterned surfactant. High local concentrations of surfactant in small discrete, spaced apart elements on a topsheet directs fluid into the absorbent core below as opposed to elongated shapes or lines that would lead to wicking of fluid within the topsheet which is not desired. It is desired to channel fluid through the topsheet, not along/within it. This improved absorption of the topsheet leads to smaller stains in absorbent articles. Smaller stains, when viewed from a wearer-facing side of the topsheet, are preferred by consumers to reassure them that the absorbent article is working and for dryness.

The patterned surfactant may cover between about 5% and about 70%, about 10% and about 60%, about 10% and about 50%, about 10% and about 40%, about 10% and about 30%, about 10% and about 20% of a total area of a garment-facing surface of the nonwoven topsheet. All % coverage areas of the patterned surfactant is measured according to the Composition Pattern Analysis Test.

The average surfactant concentration may be less than about 1% or less than about 0.5%, but greater than 0.1% of the topsheet by weight, according to NWSP 350.0 RO (15).

The concentration of surfactant within a discrete element of the patterned surfactant is greater than 1%, greater than 1.5%, but less than 10%, according to the Composition Pattern Analysis Test.

The nonwoven webs or nonwoven topsheets may comprise multicomponent fibers and at least one component of the multicomponent fibers may be bio-based. The nonwoven web or nonwoven topsheet may comprise bicomponent side-by-side continuous spunbond fibers. The nonwoven webs or nonwoven topsheets may have a basis weight in the range of about 10 gsm to about 50 gsm, about 10 gsm to about 35 gsm, about 15 gsm to about 30 gsm, or about 20 gsm to about 30 gsm, according to the Basis Weight Test herein.

### Ratios

The patterned surfactants of the present disclosure may have a ratio of a pattern spacing distance to a pattern width that is in the range of about 1.4 to about 5, or about 2 to about 3, according to the Composition Pattern Analysis Test. The patterned surfactants of the present disclosure may have a ratio of a pattern spacing distance to a pattern width that is in the range of about 1 to about 8, or about 2.5 to about 5.5, according to the Composition Pattern Analysis Test.

FIG. 33 is an example of a continuous surfactant 422 (represented by the cross hashed area) overlapped by the patterned surfactant 416 comprising a plurality of discrete, spaced apart elements for use with the nonwoven webs or nonwoven topsheets of the present disclosure. In some instances, it may be desirable to apply a continuous surfactant that overlaps the patterned surfactant or apply a patterned surface that overlaps the continuous surfactant. The patterned surfactant 416 may have a first hydrophilicity and the continuous surfactant may have a second hydrophilicity. The second hydrophilicity may be more hydrophobic than the first hydrophilicity, but may still be hydrophilic. As a result, faster bodily exudate absorption may occur where the patterned surfactant is present, but the entire zone of continuous surfactant and patterned surfactant may absorb better than portions of the nonwoven web or nonwoven topsheet not having any surfactant. These portions may be hydrophobic. Increasing overall absorption speeds and better control of bodily exudates leads to smaller viewable stains from a wearer-facing side of the topsheet which indicates to a consumer that the product is working properly.

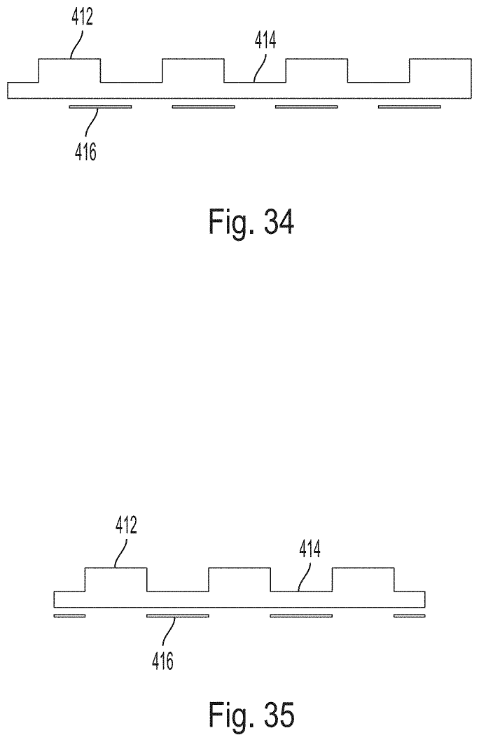

FIG. 34 is a schematic cross-sectional view of nonwoven web or nonwoven topsheet having a visually discernible pattern of three-dimensional features and with a non-registered, patterned surfactant 416 applied to a surface thereof. FIG. 35 is a schematic cross-sectional view of nonwoven web or nonwoven topsheet having a visually discernible pattern of three-dimensional features and with a registered, patterned surfactant 416 applied to a surface thereof. The surface the patterned surfactant may be applied to may be the garment-facing surface of a nonwoven topsheet. The one or more first regions are indicated as element 412 and the plurality of second regions are illustrated as element 414 in FIGS. 34 and 35. The one or more first regions 412 may have about 1.5 times, about 2 times, about 3 times, or about 4 times, as much basis weight as the plurality of second regions 414. The nonwoven web or topsheet may be made by the process described herein in regard to the structured belt or may be made using a spunlace or hydroentanglement process. In FIG. 34, the patterned surfactant 416 is not registered with the plurality of second regions 414 or discrete regions, but does at least partially overlap with the plurality of second regions 414. Due to the lower basis weight of the plurality of the second regions 414, relative to the one or more first regions 412, the patterned surfactant 416 causes fast bodily exudate absorption in the areas of overlap. In FIG. 35, the patterned surfactant 416 is registered with the plurality of second regions 414 or discrete regions. Due to the lower basis weight of the plurality of second regions 414, related to the one or more first regions 412, the patterned surfactant 416 causes fast bodily exudate wicking in the areas of overlap.

### Free Fluid Acquisition Rewet

The Free Fluid Acquisition Rewet of the absorbent articles with the visually discernible pattern of three-dimensional features and with a patterned surfactant disclosed herein may be in the range of about 0.05 grams to about 0.8 grams, about 0.05 grams to about 0.6 grams, about 0.05 grams to about 0.4 grams, or about 0.05 to about 0.55 grams, according to the Acquisition Time and Rewet Test herein.

The Free Fluid Acquisition Time of the absorbent articles with the visually discernible pattern of three-dimensional features and with a patterned surfactant disclosed herein may be in the range of about 5 seconds to about 25 seconds, about 5 seconds to about 15 seconds, or about 5 seconds to about 10 seconds, according to the Acquisition Time and Rewet Test herein.

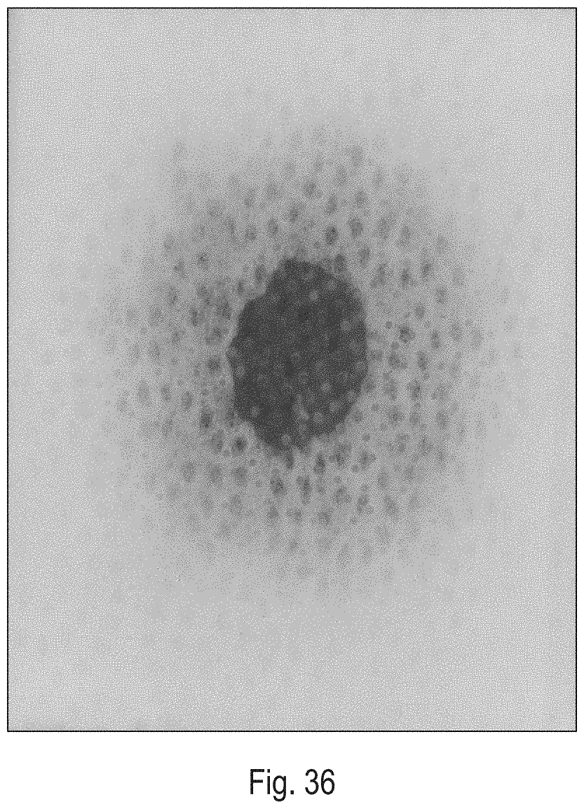

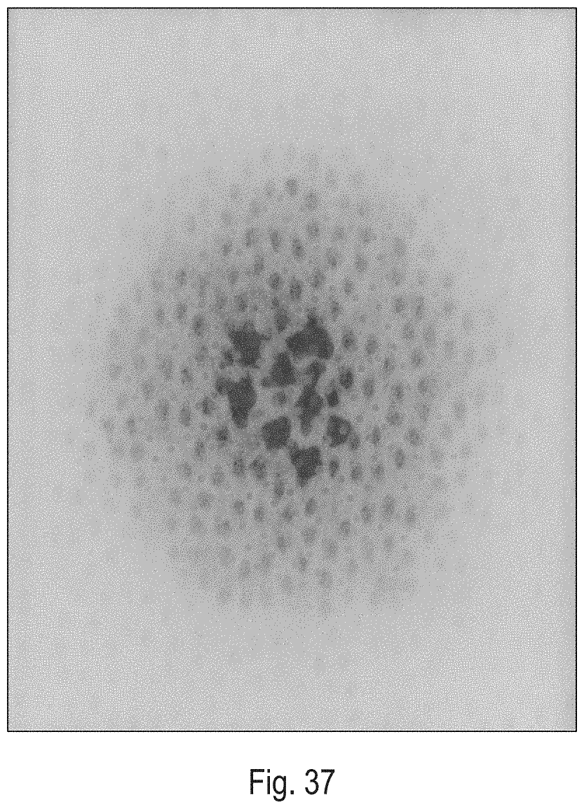

FIG. 36 is a plan view photograph of a nonwoven topsheet, and a visible stain, with a continuous surfactant applied to a garment-facing side of the nonwoven topsheet. FIG. 37 is a plan view photograph of a nonwoven topsheet, and a visible stain, with a patterned surfactant applied to a garment-facing side of the nonwoven topsheet. It is noted that the stain in FIG. 36 is much more visible than the stain in FIG. 37. Applicants attribute the reduced visibility of the stain to the patterned surfactant on the garment-facing side of the topsheet in accordance with the present disclosure.

### Nonwoven Topsheet Fiber Surface Property Manipulation

Some suitable example melt additives for fibers of the nonwoven webs or topsheets of the present disclosure are disclosed in U.S. patent application Ser. No. 16/452,903, filed on Jun. 26, 2019, P&G Docket Number 15291.

Slip agent melt additives may be included in an amount sufficient to affect and/or enhance desired haptic properties (e.g., impart a soft/silky/slick feel) to the fibers of the nonwoven topsheet. Some slip agents when melt-blended with the resin gradually migrate to the fibers surfaces during cooling or after fabrication, hence forming a thin coating with lubricating effects, in the filament surfaces. It may be desired that the slip agent be a fast-bloom slip agent, and can be a hydrocarbon having one or more functional groups selected from hydroxide, aryls and substituted aryls, halogens, alkoxys, carboxylates, esters, carbon unsaturation, acrylates, oxygen, nitrogen, carboxyl, sulfate and phosphate. In one particular form, the slip agent is a salt derivative of an aromatic or aliphatic hydrocarbon oil, notably metal salts of fatty acids, including metal salts of carboxylic, sulfuric, and phosphoric aliphatic saturated or unsaturated acid having a chain length of 7 to 26 carbon atoms, preferably 10 to 22 carbon atoms. Examples of suitable fatty acids include the monocarboxylic acids lauric acid, stearic acid, succinic acid, stearyl lactic acid, lactic acid, phthalic acid, benzoic acid, hydroxystearic acid, ricinoleic acid, naphthenic acid, oleic acid, palmitic acid, erucic acid, and the like, and the corresponding sulfuric and phosphoric acids. Suitable metals include Li, Na, Mg, Ca, Sr, Ba, Zn, Cd, Al, Sn, Pb and so forth. Representative salts include, for example, magnesium stearate, calcium stearate, sodium stearate, zinc stearate, calcium oleate, zinc oleate, magnesium oleate and so on, and the corresponding metal higher alkyl sulfates and metal esters of higher alkyl phosphoric acids.

In other examples, the slip agent may be a non-ionic functionalized compound. Suitable functionalized compounds include: (a) esters, amides, alcohols and acids of oils including aromatic or aliphatic hydrocarbon oils, for example, mineral oils, naphthenic oils, paraffinic oils; natural oils such as castor, corn, cottonseed, olive, rapeseed, soybean, sunflower, other vegetable and animal oils, and so on. Representative functionalized derivatives of these oils include, for example, polyol esters of monocarboxylic acids such as glycerol monostearate, pentaerythritol monooleate, and the like, saturated and unsaturated fatty acid amides or ethylenebis(amides), such as oleamide, erucamide, linoleamide, and mixtures thereof, glycols, polyether polyols like Carbowax, and adipic acid, sebacic acid, and the like; (b) waxes, such as carnauba wax, microcrystalline wax, polyolefin waxes, for example polyethylene waxes; (c) fluoro-containing polymers such as polytetrafluoroethylene, fluorine oils, fluorine waxes and so forth; and (d) silicon compounds such as silanes and silicone polymers, including silicone oils, polydimethylsiloxane, amino-modified polydimethylsiloxane, and so on.

Fatty amides that may be useful for purposes of the present disclosure are represented by the formula: RC(O)NHR¹, where R is a saturated or unsaturated alkyl group having 7 to 26 carbon atoms, preferably 10 to 22 carbon atoms, and R1 is independently hydrogen or a saturated or unsaturated alkyl group having from 7 to 26 carbon atoms, preferably 10 to 22 carbon atoms. Compounds according to this structure include for example, palmitamide, stearamide, arachidamide, behenamide, oleamide, erucamide, linoleamide, stearyl stearamide, palmityl palmitamide, stearyl arachidamide and mixtures thereof.

Ethylenebis(amides) that may be useful for purposes of the present disclosure are represented by the formula:

RC(O)NHCH₂CH₂NHC(O)R,

where each R is independently is a saturated or unsaturated alkyl group having 7 to 26 carbon atoms, preferably 10 to 22 carbon atoms. Compounds according to this structure include for example, stearamidoethylstearamide, stearamidoethylpalmitamide, palmitamidoethylstearamide, ethylenebisstearamide, ethylenebisoleamide, stearylerucamide, erucamidoethylerucamide, oleamidoethyloleamide, erucamidoethyloleamide, oleamidoethylerucamide, stearamidoethylerucamide, erucamidoethylpalmitamide, palmitamidoethyloleamide and mixtures thereof.

Commercially available examples of fatty amides include Ampacet 10061 (Ampacet Corporation, White Plains, N.Y., USA) which comprises 5 percent of a 50:50 mixture of the primary amides of erucic and stearic acids in polyethylene; Elvax 3170 (E.I. du Pont de Nemours and Company/DuPont USA, Wilmington, Del., USA) which comprises a similar blend of the amides of erucic and stearic acids in a blend of 18 percent vinyl acetate resin and 82 percent polyethylene. Slip agents also are available from Croda International Plc (Yorkshire, United Kingdom), including Crodamide OR (an oleamide), Crodamide SR (a stearamide), Crodamide ER (an erucamide), and Crodamide BR (a behenamide); and from Crompton, including Kemamide S (a stearamide), Kemamide B (a behenamide), Kemamide O (an oleamide), Kemamide E (an erucamide), and Kemamide (an N,N′-ethylenebisstearamide). Other commercially available slip agents include Erucamid ER erucamide.

Nonwoven webs within contemplation of the present disclosure may include slip agents/softness melt additives independently, or in conjunction with other additives that affect the surface energy (hydrophilicity/hydrophobicity), or in conjunction with other fiber feature variations including but not limited to fiber size, filament cross-sectional shape, fiber cross-sectional configuration, and/or curled fiber variations. For examples of nonwoven web materials including two or more web layers, or two or more deposited layers of differing fiber, additives may be included in fiber of one layer but not the other, or differing additives may be included in fibers of differing layers.

In some examples, a hydrophobizing melt additive may be added directly or as master batch to the polymer melt during the spinning process. Suitable melt additives may include, for example, lipid esters or polysiloxanes. When a hydrophobizing melt additive is blended into resin(s), the additive in the resulting spun filament can bloom to its external surface and create a film covering portions of the surface, form fibrils, flakes, particles, or other surface features that have low surface energy.

Any suitable hydrophobizing melt additive may be utilized. Examples of hydrophobizing melt additives include fatty acids and fatty acid derivatives. The fatty acids may originate from vegetable, animal, and/or synthetic sources. Some fatty acids may range from a C8 fatty acid to a C30 fatty acid, or from a C12 fatty acid to a C22 fatty acid. In other forms, a substantially saturated fatty acid may be used, particularly when saturation arises as a result of hydrogenation of fatty acid precursor. Examples of fatty acid derivatives include fatty alcohols, fatty acid esters, and fatty acid amides. Suitable fatty alcohols (R—OH) include those derived from C12-C28 fatty acids.

Suitable fatty acid esters include those fatty acid esters derived from a mixture of C12-C28 fatty acids and short chain (C1-C8, preferably C1-C3) monohydric alcohols preferably from a mixture of C12-C22 saturated fatty acids and short chain (C1-C8, preferably C1-C3) monohydric alcohols. The hydrophobizing melt additive may comprise a mixture of mono, di, and/or tri-fatty acid esters. An example includes fatty acid ester with glycerol as the backbone as illustrated in illustration [1], below:

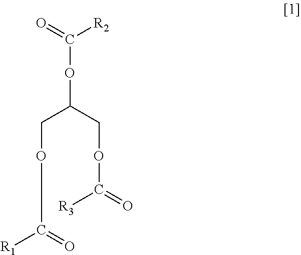

where R1, R2, and R3 each is an alkyl ester having carbon atoms ranging from 11 to 29. In some forms, the glycerol derived fatty acid ester has at least one alkyl chain, at least two, or three chains to a glycerol, to form a mono, di, or triglyceride. Suitable examples of triglycerides include glycerol thibehenate, glycerol tristearate, glycerol tripalmitate, and glycerol trimyristate, and mixtures thereof. In the case of triglycerides and diglycerides, the alkyl chains could be the same length, or different length. Example includes a triglyceride with one alkyl C18 chain and two C16 alkyl chain, or two C18 alkyl chains and one C16 chain. Preferred triglycerides include alkyl chains derived from C14-C22 fatty acids.

Other suitable hydrophobizing melt additives include hydrophobic silicones. Additional suitable hydrophobizing melt additives are disclosed in U.S. patent application Ser. No. 14/849,630 and U.S. patent application Ser. No. 14/933,028. Another suitable hydrophobizing melt additive is available from Techmer PM in Clinton, Tenn. under the trade name PPM17000 High Load Hydrophobic. One specific example of a hydrophobizing melt additive is glycerol tristearate. As used herein, glycerol tristearate is defined as a mixture of long-chained triglycerides containing predominately C18 and C16 saturated alkyl chain lengths. Additionally, there could be varying degrees of unsaturation and cis to trans unsaturated bond configurations. The alkyl chain lengths could range from about C10 to about C22. The degrees of unsaturation typically will range from 0 to about 3 double bonds per alkyl chain. The ratio of cis to trans unsaturated bond configurations can range from about 1:100 to about 100:1. Other suitable examples for use with polypropylene and/or polyethylene, a triglyceride which contains either stearic acid or palmic acid or both as the fatty acid components, or a mixture of such triglycerides. Other suitable hydrophobizing or hydrophobic melt additives may comprise erucamide or polysiloxanes.

Any suitable hydrophilizing additive can be used. Some suitable examples include those available from Techmer PM, Clinton, Tenn. sold under the trade name of TECHMER PPM15560; TPM12713, PPM19913, PPM 19441, PPM19914, PPM112221 (for polypropylene), PM19668, PM112222 (for polyethylene). Additional examples are available from Polyvel Inc. located in Hammonton, N.J., sold under the trade name of POLYVEL VW351 PP Wetting Agent (for polypropylene); from Goulston Technologies Inc. located in Monroe, N.C. sold under the trade name HYDROSORB 1001; as well as those hydrophilizing additives disclosed in U.S. Patent Application Publication No. 2012/0077886 and U.S. Pat. Nos. 5,969,026 and 4,578,414.

### Test Methods

### Air Permeability Test

The Air Permeability Test is used to determine the level of air flow in cubic feet per minute (cfm) through a forming belt. The Air Permeability Test is performed on a Texas Instruments model FX3360 Portair Air Permeability Tester, available from Textest AG, Sonnenbergstrasse 72, CH 8603 Schwerzenbach Switzerland. The unit utilizes a 20.7 mm orifice plate for air permeability ranges between 300-1000 cfm. If air permeability is lower than 300 cfm the orifice plate needs to be reduced; if higher than 1000 cfm the orifice plate needs to be increased. Air permeability can be measured in localized zones of a forming belt to determine differences in air permeability across a forming belt.

### Test Procedure

1. Power on the FX3360 instrument.

2. Select a pre-determined style having the following setup:
a. Material: Standard
b. Measurement Property: Air Permeability (AP)
c. Test Pressure: 125 Pa (pascals)
d. T-factor: 1.00
e. Test point pitch: 0.8 inch.

3. Position the 20.7 mm orifice plate on the top side of the forming belt(the side with the three-dimensional protrusions) at the position of interest.

4. Selecting “Spot Measurement” on the touch screen of the testing unit.

5. Reset the sensor prior to measurement, if necessary.

6. Once reset, select the “Start” button to begin measurement.

7. Wait until the measurement stabilizes and record the cfm reading on the screen.

8. Select, the “Start” button again to stop measurement.

### Basis Weight Test

Basis weight of the nonwoven webs or nonwoven topsheets described herein may be determined by several available techniques, but a simple representative technique involves taking an absorbent article or other consumer product, removing any elastic which may be present and stretching the absorbent article or other consumer product to its full length. A punch die having an area of 45.6 cm² is then used to cut a piece of the nonwoven web (e.g., topsheet, outer cover) from the approximate center of the absorbent article or other consumer product in a location which avoids to the greatest extent possible any adhesive which may be used to fasten the nonwoven web to any other layers which may be present and removing the nonwoven web from other layers (using cryogenic spray, such as Cyto-Freeze, Control Company, Houston, Texas, if needed). The sample is then weighed and dividing by the area of the punch die yields the basis weight of the nonwoven web or nonwoven topsheet. Results are reported as a mean of 5 samples to the nearest 0.1 gram per square meter (gsm).

### Emtec Test

The Emtec Test is performed on portions of nonwoven webs of interest. In this test, TS7, TS750, and D values are measured using an Emtec Tissue Softness Analyzer (“Emtec TSA”) (Emtec Electronic GmbH, Leipzig, Germany) interfaced with a computer running Emtec TSA software (version 3.19 or equivalent). The Emtec TSA includes a rotor with vertical blades which rotate on the test sample at a defined and calibrated rotational speed (set by manufacturer) and contact force of 100 mN. Contact between the vertical blades and the test sample creates vibrations both in the blades and in the test piece, and the resulting sound is recorded by a microphone within the instrument. The recorded sound file is then analyzed by the Emtec TSA software to determine TS7 and TS750 values. The D value is a measure of sample stiffness and is based on the vertical distance required for the contact force of the blades on test sample to be increased from 100 mN to 600 mN. The sample preparation, instrument operation, and testing procedures are performed according the instrument manufacturer's specifications. Sample Preparation

A test sample is prepared by cutting a square or circular portion of interest from a nonwoven web of an absorbent article. It is preferable that freeze spray is not used to remove the nonwoven web to be analyzed from the absorbent article, though it is acceptable to use freeze spray in a distal region to aid in initiating the separation of layers. Test samples are cut to a length and width (diameter in the case of a circular sample) of no less than about 90 mm and no greater than about 120 mm to ensure the sample can be clamped into the TSA instrument properly. (If an absorbent article does not contain a sufficiently large area of the substrate of interest to extract a sample of the size specified above, it is acceptable to sample equivalent material from roll stock.) Test samples are selected to avoid unusually large creases or folds within the testing region. Six substantially similar replicate samples are prepared for testing.

All samples are equilibrated at TAPPI standard temperature and relative humidity conditions (23° C.±2° C. and 50%±2%) for at least 2 hours prior to conducting the TSA testing, which is also conducted under TAPPI conditions.

### Testing Procedure

The instrument is calibrated according to the Emtec's instructions using the 1-point calibration method with the appropriate reference standards (so-called “ref.2 samples,” or equivalent, available from Emtec).

A test sample is mounted in the instrument with the surface of interest facing upward, and the test is performed according to the manufacturer's instructions. The software displays values for TS7, TS750, and D when the automated instrument testing routine is complete. TS7 and TS750 are each recorded to the nearest 0.01 dB V² rms, and D is recorded to the nearest 0.01 mm/N. The test sample is then removed from the instrument and discarded. This testing procedure is performed individually on the corresponding surfaces of interest of each of the six of the replicate samples (wearer-facing surface for topsheet samples and garment-facing surface for outer cover nonwoven material samples).

The value of TS7, TS750, and D are each averaged (arithmetic mean) across the six sample replicates. The average values of TS7 and TS750 are reported to the nearest 0.01 dB V² rms. The average value of D is reported to the nearest 0.01 mm/N.

### Micro-CT Intensive Property Measurement Test

The micro-CT intensive property measurement method measures the basis weight, thickness and volumetric density values within visually discernable regions of a substrate sample. It is based on analysis of a 3D x-ray sample image obtained on a micro-CT instrument (a suitable instrument is the Scanco μCT50 available from Scanco Medical AG, Switzerland, or equivalent). The micro-CT instrument is a cone beam microtomograph with a shielded cabinet. A maintenance free x-ray tube is used as the source with an adjustable diameter focal spot. The x-ray beam passes through the sample, where some of the x-rays are attenuated by the sample. The extent of attenuation correlates to the mass of material the x-rays have to pass through. The transmitted x-rays continue on to the digital detector array and generate a 2D projection image of the sample. A 3D image of the sample is generated by collecting several individual projection images of the sample as it is rotated, which are then reconstructed into a single 3D image. The instrument is interfaced with a computer running software to control the image acquisition and save the raw data. The 31) image is then analyzed using image analysis software (a suitable image analysis software is MATLAB available from The Mathworks, Inc., Natick, Mass., or equivalent) to measure the basis weight, thickness and volumetric density intensive properties of regions within the sample. Sample Preparation:

To obtain a sample for measurement, lay a single layer of the dry substrate material out flat and die cut a circular piece with a diameter of 30 mm.

If the substrate material is a layer of an absorbent article, for example a topsheet, backsheet nonwoven, acquisition layer, distribution layer, or other component layer; tape the absorbent article to a rigid flat surface in a planar configuration, Carefully separate the individual substrate layer from the absorbent article. A scalpel and/or cryogenic spray (such as Cyto-Freeze, Control Company, Houston Tex.) can be used to remove a substrate layer from additional underlying layers, if necessary, to avoid any longitudinal and lateral extension of the material. Once the substrate layer has been removed from the article proceed with die cutting the sample as described above.

If the substrate material is in the form of a wet wipe, open a new package of wet wipes and remove the entire stack from the package. Remove a single wipe from the middle of the stack, lay it out flat and allow it to dry completely prior to die cutting the sample for analysis.

A sample may be cut from any location containing the visually discernible zone to be analyzed. Within a zone, regions to be analyzed are ones associated with a three-dimensional feature defining a microzone. The microzone comprises a least two visually discernible regions. A zone, three-dimensional feature, or microzone may be visually discernable due to changes in texture, elevation, or thickness. Regions within different samples taken from the same substrate material may be analyzed and compared to each other. Care should be taken to avoid folds, wrinkles or tears when selecting a location for sampling.

### Image Acquisition:

Set up and calibrate the micro-CT instrument according to the manufacturer's specifications. Place the sample into the appropriate holder, between two rings of low density material, which have an inner diameter of 25 mm. This will allow the central portion of the sample to lay horizontal and be scanned without having any other materials directly adjacent to its upper and lower surfaces. Measurements should be taken in this region. The 3D image field of view is approximately 35 mm on each side in the xy-plane with a resolution of approximately 5000 by 5000 pixels, and with a sufficient number of 7 micron thick slices collected to fully include the z-direction of the sample. The reconstructed 3D image resolution contains isotropic voxels of 7 microns. Images are acquired with the source at 45 kVp and 133 μA with no additional low energy filter. These current and voltage settings may be optimized to produce the maximum contrast in the projection data with sufficient x-ray penetration through the sample, but once optimized held constant for all substantially similar samples. A total of 1500 projections images are obtained with an integration time of 1000 ms and 3 averages. The projection images are reconstructed into the 3D image, and saved in 16-bit RAW format to preserve the full detector output signal for analysis.

### Image Processing:

Load the 3D image into the image analysis software. Threshold the 3D image at a value which separates, and removes, the background signal due to air, but maintains the signal from the sample fibers within the substrate.

Three 2D intensive property images are generated from the thresheld 3D image. The first is the Basis Weight Image. To generate this image, the value for each voxel in an xy-plane slice is summed with all of its corresponding voxel values in the other z-direction slices containing signal from the sample. This creates a 2.D image where each pixel now has a value equal to the cumulative signal through the entire sample.

In order to convert the raw data values in the Basis Weight Image into real values a basis weight calibration curve is generated. Obtain a substrate that is of substantially similar composition as the sample being analyzed and has a uniform basis weight. Follow the procedures described above to obtain at least ten replicate samples of the calibration curve substrate. Accurately measure the basis weight, by taking the mass to the nearest 0.0001 g and dividing by the sample area and converting to grams per square meter (gsm), of each of the single layer calibration samples and calculate the average to the nearest 0.(>1 gsm. Following the procedures described above, acquire a micro-CT image of a single layer of the calibration sample substrate. Following the procedure described above process the micro-CT image, and generate a Basis Weight Image containing raw data values. The real basis weight value for this sample is the average basis weight value measured on the calibration samples. Next, stack two layers of the calibration substrate samples on top of each other, and acquire a micro-CT image of the two layers of calibration substrate. Generate a basis weight raw data image of both layers together, whose real basis weight value is equal to twice the average basis weight value measured on the calibration samples. Repeat this procedure of stacking single layers of the calibration substrate, acquiring a micro-CT image of all of the layers, generating a raw data basis weight image of all of the layers, the real basis weight value of which is equal to the number of layers times the average basis weight value measured on the calibration samples. A total of at least four different basis weight calibration images are obtained. The basis weight values of the calibration samples must include values above and below the basis weight values of the original sample being analyzed to ensure an accurate calibration. The calibration curve is generated by performing a linear regression on the raw data versus the real basis weight values for the four calibration samples. This linear regression must have an R2 value of at least 0.95, if not repeat the entire calibration procedure. This calibration curve is now used to convert the raw data values into real basis weights.

The second intensive property 2D image is the Thickness Image. To generate this image the upper and lower surfaces of the sample are identified, and the distance between these surfaces is calculated giving the sample thickness. The upper surface of the sample is identified by starting at the uppermost z-direction slice and evaluating each slice going through the sample to locate the z-direction voxel for all pixel positions in the xy-plane where sample signal was first detected. The same procedure is followed for identifying the lower surface of the sample, except the z-direction voxels located are all the positions in the xy-plane where sample signal was last detected. Once the upper and lower surfaces have been identified they are smoothed with a 15×15 median filter to remove signal from stray fibers. The 2D Thickness Image is then generated by counting the number of voxels that exist between the upper and lower surfaces for each of the pixel positions in the xy-plane. This raw thickness value is then converted to actual distance, in rrricrons, by multiplying the voxel count by the 7 μm slice thickness resolution.

The third intensive property 2D image is the Volumetric Density Image. To generate this image divide each xy-plane pixel value in the Basis Weight Image, in units of gsm, by the corresponding pixel in the Thickness Image, in units of microns. The units of the Volumetric Density Image are grams per cubic centimeter (g/cc).

Micro-CT Basis Weight, Thickness and Volumetric Density Intensive, Properties:
Begin by identifying the region to be analyzed. A region to be analyzed is one associated with a three-dimensional feature defining a microzone. The microzone comprises a least two visually discernible regions. A zone, three-dimensional feature, or microzone may be visually discernable due to changes in texture, elevation, or thickness. Next, identify the boundary of the region to be analyzed. The boundary of a region is identified by visual discernment of differences in intensive properties when compared to other regions within the sample. For example, a region boundary can be identified based by visually discerning a thickness difference when compared to another region in the sample. Any of the intensive properties can be used to discern region boundaries on either the physical sample itself of any of the micro-CT intensive property images. Once the boundary of the region has been identified, draw an oval or circular “region of interest” (ROI) within the interior of the region. The ROI should have an area of at least 0.1 mm2, and be selected to measure an area with intensive property values representative of the identified region. From each of the three intensive property images calculate the average basis weight, thickness and volumetric density within the ROI. Record these values as the region's basis weight to the nearest 0.01 gsm, thickness to the nearest 0.1 micron and volumetric density to the nearest 0.0001 g/cc.

### Artificial Menstrual Fluid (AMF) Preparation

The Artificial Menstrual Fluid (AMF) is composed of a mixture of defibrinated sheep blood, a phosphate buffered saline solution and a mucous component. The AMF is prepared such that it has a viscosity between 7.15 to 8.65 centistokes at 23° C.

Viscosity on the AMF is performed using a low viscosity rotary viscometer (a suitable instrument is the Cannon LV-2020 Rotary Viscometer with UL adapter, Cannon Instrument Co., State College, Pa., or equivalent). The appropriate size spindle for the viscosity range is selected, and instrument is operated and calibrated as per the manufacturer. Measurements are taken at 23° C.±1° C. and at 60 rpm. Results are reported to the nearest 0.01 centistokes.

Reagents needed for the AMF preparation include: defibrinated sheep blood with a packed cell volume of 38% or greater (collected under sterile conditions, available from Cleveland Scientific, Inc., Bath, Ohio, or equivalent), gastric mucin with a viscosity target of 3-4 centistokes when prepared as a 2% aqueous solution (crude form, available from Sterilized American Laboratories, Inc., Omaha, Nebr., or equivalent), 10% v/v lactic acid aqueous solution, 10% w/v potassium hydroxide aqueous solution, sodium phosphate dibasic anhydrous (reagent grade), sodium chloride (reagent grade), sodium phosphate monobasic monohydrate (reagent grade) and distilled water, each available from VWR International or equivalent source.

The phosphate buffered saline solution consists of two individually prepared solutions (Solution A and Solution B). To prepare 1 L of Solution A, add 1.38±0.005 g of sodium phosphate monobasic monohydrate and 8.50±0.005 g of sodium chloride to a 1000 mL volumetric flask and add distilled water to volume. Mix thoroughly. To prepare 1 L of Solution B, add 1.42±0.005 g of sodium phosphate dibasic anhydrous and 8.50±0.005 g of sodium chloride to a 1000 mL volumetric flask and add distilled water to volume. Mix thoroughly. To prepare the phosphate buffered saline solution, add 450±10 mL of Solution B to a 1000 mL beaker and stir at low speed on a stir plate. Insert a calibrated pH probe (accurate to 0.1) into the beaker of Solution B and add enough Solution A, while stirring, to bring the pH to 7.2±0.1.

The mucous component is a mixture of the phosphate buffered saline solution, potassium hydroxide aqueous solution, gastric mucin and lactic acid aqueous solution. The amount of gastric mucin added to the mucous component directly affects the final viscosity of the prepared AMF. To determine the amount of gastric mucin needed to achieve AMF within the target viscosity range (7.15-8.65 centistokes at 23° C.) prepare 3 batches of AMF with varying amounts of gastric mucin in the mucous component, and then interpolate the exact amount needed from a concentration versus viscosity curve with a least squares linear fit through the three points. A successful range of gastric mucin is usually between 38 to 50 grams.

To prepare about 500 mL of the mucous component, add 460±10 mL of the previously prepared phosphate buffered saline solution and 7.5±0.5 mL of the 10% w/v potassium hydroxide aqueous solution to a 1000 mL heavy duty glass beaker. Place this beaker onto a stirring hot plate and while stirring, bring the temperature to 45° C.±5 C°. Weigh the pre-determined amount of gastric mucin (±0.50 g) and slowly sprinkle it, without clumping, into the previously prepared liquid that has been brought to 45° C. Cover the beaker and continue mixing. Over a period of 15 minutes bring the temperature of this mixture to above 50° C. but not to exceed 80° C. Continue heating with gentle stirring for 2.5 hours while maintaining this temperature range. After the 2.5 hours has elapsed, remove the beaker from the hot plate and cool to below 40° C. Next add 1.8±0.2 mL of the 10% v/v lactic acid aqueous solution and mix thoroughly. Autoclave the mucous component mixture at 121° C. for 15 minutes and allow 5 minutes for cool down. Remove the mixture of mucous component from the autoclave and stir until the temperature reaches 23° C.±1C°.

Allow the temperature of the sheep blood and mucous component to come to 23° C.±1° C. Using a 500 mL graduated cylinder, measure the volume of the entire batch of the previously prepared mucous component and add it to a 1200 mL beaker. Add an equal volume of sheep blood to the beaker and mix thoroughly. Using the viscosity method previously described, ensure the viscosity of the AMF is between 7.15-8.65 centistokes. If not the batch is disposed and another batch is made adjusting the mucous component as appropriate.

The qualified AMF should be refrigerated at 4° C. unless intended for immediate use. AMF may be stored in an air-tight container at 4° C. for up to 48 hours after preparation. Prior to testing, the AMF must be brought to 23° C.±1° C. Any unused portion is discarded after testing is complete.

### Acquisition Time and Rewet Test

Acquisition time is measured for an absorbent article loaded with Artificial Menstrual Fluid (AMF), prepared as described herein. A known volume of AMF is introduced three times, each successive dose starting two minutes after the previous dose has absorbed. The time required for each dose to be absorbed by the article are recorded. Subsequent to the acquisition test, a rewet method is performed to determine the mass of fluid expressed from the article under pressure. Test samples are conditioned at 23° C.±2° C. and 50%±2% relative humidity for 2 hours prior to testing and all testing is performed under these same environmental conditions.

The confining weight used for the rewet test has a flat level base with a contact surface that is 64±1 mm wide by 83±1 mm and a mass of 2268±2 grams (5 pounds). This weight provides a confining pressure of 4.1 kPa (0.60 psi) on the test article. The rewet substrate is two sheets of filter paper with dimensions 4 inch by 4 inch. A suitable filter paper is Ahlstrom Grade 989 (available from Ahlstrom-Munksjo North America LLC, Alpharetta, Ga.) or equivalent.

Perform the acquisition test as follows. Remove the test article from its wrapper. If folded, gently unfold and smooth out any wrinkles. Place the test article horizontally flat, with the top sheet of the product facing upward. Position the tip of a mechanical pipette about 1 cm above the center (longitudinal and lateral midpoint) of the article's absorbent core, and accurately pipette 1.00 mL±0.05 mL of AMF onto the surface. The fluid is dispensed without splashing, within a period of 2 seconds. As soon as the fluid makes contact with the test sample, start a timer accurate to 0.01 seconds. After the fluid has been acquired (no pool of fluid left on the surface), stop the timer and record the acquisition time to the nearest 0.01 second. Wait 2 minutes. In like fashion, a second and third dose of AMF are applied to the test sample and the acquisition times are recorded to the nearest 0.01 second. Proceed with the Rewet test 2 minutes after the third dose has been acquired.

Perform the rewet part of the test as follows. Measure the dry mass of two filter papers to the nearest 0.0001 grams and record as Mass_{Dry}. Gently place the dry filter papers over the center (longitudinal and lateral midpoint) of the test article's absorbent core. Gently place the base of the confining weight over the center (longitudinal and lateral midpoint) of the filter paper, positioning the length (long side) of the weight parallel to the longitudinal direction of the test article. Immediately start a timer accurate to 0.01 seconds. After 30 seconds, carefully remove the confining weight. Measure the mass of the filter papers to the nearest 0.0001 grams and record as Mass_{Set}. Calculate rewet as the difference between Mass_{Wet} and Mass_{Dry} for the filter papers and record as Rewet Value to the nearest 0.0001 grams.

This entire procedure is repeated on five substantially similar replicate articles. The reported value is the average of the five individual recorded measurements for each Acquisition Time (first, second and third) to the nearest 0.01 second and Rewet Value to the nearest 0.0001 gram.

### Composition Pattern Analysis Test

To determine the presence of a composition pattern (e.g. patterned surfactant) on the outermost body facing layer (i.e. topsheet) of an absorbent article, the layer is excised from the absorbent article and placed on the surface of colored water causing any composition pattern to exhibit the color of the water. If a composition pattern is observed, a photographic image is captured and further analysis is performed to measure the width and spacing of the discrete objects making up the composition pattern using image analysis. Test specimens are conditioned at 23° C.±2° C. and 50%±2% relative humidity for 2 hours prior to testing and all testing is performed under these same environmental conditions.

A fresh absorbent article, within 6 months of the date of production, is obtained. The absorbent article is removed from its wrapper, if present, and a mark is made on the topsheet 3 mm inboard from each longitudinal end along the longitudinal axis. The distance between the two marks is measured and recorded as the gage length to the nearest 1 mm. To obtain a test specimen, the entire topsheet is excised from the article, using care to not impart any contamination or distortion to the layer during the process. A cryogenic spray (such as Quick-Freeze, Miller-Stephenson Company, Danbury, Conn.) may be used to remove the test specimen from the underlying layers if necessary. A test liquid is prepared by adding 0.05 wt % methylene blue dye (available from VWR International), or equivalent, to deionized water. The test specimen is exposed to the colored test liquid as follows.

A shallow dish is obtained that is large enough to allow the entire test specimen to lie horizontally flat inside. A total of 6 rectangular bars are obtained that are approximately 3 mm thick, 25 mm wide, and with a length equivalent to the width (lateral edge to lateral edge) of the test specimen at the gage marks. The bars are made of stainless steel (or equivalent) and heavy enough to sufficiently hold the test specimen in place. The test specimen is attached to two of the bars. Two bars are used as risers in the dish of liquid and the other two bars are used as risers in the light box.

The test specimen is placed on a horizontally flat surface with the garment side facing up. Using double sided tape that is about 3 mm wide, secure the test specimen to the bottom surface of two bars immediately outboard of the two gage marks. The distance between the test specimen bars is adjusted such that the distance between them is equal to the gage length. During subsequent handling of the test specimen, use care at all times to avoid twisting or stretching the test specimen beyond the gage length. One riser is placed at each end of the shallow dish such that the distance between them is equal to the gage length. The dish is filled with the colored test liquid to a depth equal to the height of the risers. The test specimen is transferred to the dish of colored test liquid and the bars placed onto the risers in the dish such that the body facing surface of the test specimen makes contact with the surface of the colored test liquid. If the test specimen has a composition pattern present it will become notably colored (e.g. blue) within 10 seconds due to wetting by the colored test liquid, and the test proceeds. If a composition pattern is not observed on the specimen the test is discontinued. After 10 seconds, if a composition pattern is observed, the test specimen is transferred (still attached to two bars) from the colored liquid to a sheet of blotting paper (e.g. Whatman grade 1, available from VWR International) that is the same size or larger than the test specimen. The body facing surface of the test specimen is allowed to make contact with the blotting paper for no more than 3 seconds to remove any droplets of test liquid from the back surface.

Without undue delay the test specimen is transferred into a light box that provides stable uniform lighting evenly across the entire base of the light box. A suitable light box is the Sanoto MK50 (Sanoto, Guangdong, China), or equivalent, which provides an illumination of 5500 lux at a color temperature of 5500K. The illumination and color temperature are verified using a light meter prior to capturing images inside the light box to ensure the lighting conditions are consistent between each image obtained. A suitable light meter is the CL-70F CRI Illuminance Meter available from Konica Minolta, or equivalent. Two riser bars are placed on a matte white surface inside the bottom of the light box such that the distance between them is equal to the gage length. The specimen bars are placed onto the risers, thereby suspending the specimen horizontally flat over the matte white surface.

A digital single-lens reflex (DSLR) camera with manual setting controls (e.g. a Nikon D4OX available from Nikon Inc., Tokyo, Japan, or equivalent) is mounted directly above an opening in the top of the light box so that the entire test specimen is visible within the camera's field of view.

Using a standard 18% gray card (e.g., Kodak Gray Card R-27 with a Munsell 18% Reflectance (Gray) Neutral Patch, available from X-Rite; Grand Rapids, MI, or equivalent) the camera's white balance is custom set for the lighting conditions inside the light box. The camera's manual settings are set so that the image is properly exposed such that there is no signal clipping due to saturation in any of the color channels. Suitable settings might be an aperture setting of f/11, an ISO setting of 400, a shutter speed setting of 1/400 sec., and an approximate focal length of 35 mm. The camera is mounted approximately 14 inches directly above the specimen. The image is properly focused, captured, and saved as a 24 bit (8 bits per channel) RGB color JPEG file. The resulting image must contain the entire test specimen at a minimum resolution of 15 pixels/mm. A photographic image of the entire test specimen is captured. The test specimen is removed from the light box. A distance scale (certified by NIST) is placed horizontally flat on top of the risers inside of the light box, and a calibration image is captured with the same camera settings and under the same lighting conditions as those used for the test specimen image.

### Pattern Object Dimension Measurements:

Pattern images are spatially calibrated and analyzed using image analysis software (a suitable software is MATLAB, available from The Mathworks, Inc, Natick, MA, or equivalent). The calibration image is opened in the image analysis program and a linear distance calibration is performed using the distance scale captured in the calibration image. The test specimen image is opened in the image analysis program and the distance scale is set using the distance calibration to determine the number of pixels per millimeter. The RGB color pattern image is then converted to an 8 bit grayscale according to the following weighted sum of the R, G, and B components, where the gray level is rounded to the nearest integer value.

Gray Level=0.2989×R+0.5870×G+0.1140×B

A 5×5 pixel median filter is applied to the image to remove noise, followed by a 5×5 pixel mean filter to smooth the image. The 8-bit grayscale image is then converted to a binary image by thresholding using Otsu's method, which calculates the threshold level that minimizes the weighted intra-class variance between foreground and background pixels. The discrete objects corresponding to the patterned surfactant in the binary image are identified with foreground pixels, and are assigned a value of 1 (one) while background pixels are assigned a value of 0 (zero). The individual objects in the binary image may contain bridging pixels that connect objects not apparently intended to be connected in the pattern. The foreground pattern objects are eroded enough times to separate patterned objects intended to be discrete in the pattern using a 3×3 square structuring element. This erosion operation removes any foreground pixel that is touching (an 8-connected neighbor to every pixel that touches one of their edges or corners) a background pixel, thereby removing a layer of pixels around the periphery of the patterned object. Using a 3×3 square structuring element, a dilation operation is then performed an equivalent number of times to restore the patterned objects to their original dimensions. This dilation operation converts any background pixel that is touching (8-connected neighbor) a foreground pixel into a foreground pixel, thereby adding a layer of pixels around the periphery of the patterned object. Holes within the patterned objects not apparently intended to be part of the pattern are closed by performing dilation operations a sufficient number of times to close holes within objects, followed by an equivalent number of iterations of erosion operations to restore the original dimensions of the object.

A connected components (8-connected neighbor) operation is utilized to identify all of the individual patterned objects. This connected components algorithm is executed on the binary image, which groups, or clusters, together the foreground pixels that are 8-connected (touching one of their edges or corners) to neighboring foreground pixels. Any remaining foreground pixel clusters that are not part of the regular pattern are removed or excluded from further analysis. The centroid of each patterned object is identified and its (x,y) coordinate location recorded.

Each of the discrete identified patterned objects is analyzed using the image analysis software. All the individual patterned objects areas, perimeters, maximum feret diameters (length of the apertures), minimum feret diameters (width of the apertures), and centroid locations are measured and recorded. Individual patterned object areas are recorded to the nearest 0.01 mm², patterned object perimeters and feret diameters (length and width), to the nearest 0.01 mm. The total number of patterned objects is recorded. The number of patterned objects identified is divided by the projected area of the test specimen in the image, and this quotient is recorded as the patterned object Density value to the nearest 0.1 patterned objects per cm². In addition to these measurements, the Aspect Ratio, defined for each patterned object as the quotient of its length divided by its width, is calculated and recorded. The statistical mean (average) of all the recorded individual patterned object values for each of the dimension measurements, including pattern width, are calculated and reported.

### Pattern Percent Area Measurement:

All the recorded individual patterned object areas are summed. This sum is then divided by the projected area of the test specimen in the image. This value is multiplied by 100% and reported as the percent area of coverage to the nearest 0.1%.

### Pattern Spacing Measurement:

Using the recorded location of each patterned object's centroid, the Euclidian distance from each patterned object's centroid to all of the other patterned object centroids is calculated. For each patterned object, the shortest distance is identified and recorded as the nearest neighbor distance. Any spurious distance values that are not representative of the patterned objects within the pattern are excluded. The arithmetic mean nearest neighbor distance value for all of the patterned objects within the pattern image is calculated and reported as the pattern spacing distance to the nearest 0.1 mm.

Concentration of surfactant within a discrete element of the Patterned Surfactant:

This is calculated according to average surfactant concentration divided by pattern percent area measurement and reported to the nearest 0.1%.

### Examples/Combinations:

1. An absorbent article comprising:

a nonwoven topsheet;

a liquid impermeable backsheet;

an absorbent core positioned at least partially intermediate the topsheet and the backsheet;

the nonwoven topsheet comprising:
a first surface;
a second surface; and
a visually discernible pattern of three-dimensional features on the first surface or the second surface, wherein the three-dimensional features comprise one or more first regions and a plurality of second regions;
wherein the one or more first regions have a first value of an average intensive property, wherein the plurality second regions have a second value of the average intensive property, wherein the first value is greater than the second value, and wherein the first value and the second value are greater than zero;
wherein the first regions are continuous;
wherein the second regions are discrete, and wherein at least some of the first regions surround at least some of the second regions;
a patterned surfactant on a garment-facing surface of the nonwoven topsheet;
wherein the patterned surfactant comprises a plurality of discrete, spaced apart elements; and
wherein the discrete, spaced apart elements have an area between about 0.75 mm² and 30 mm², preferably between about 0.75 mm² to about 15 mm², according to the Composition Pattern Analysis Test.
2. The absorbent article according to paragraph 1, wherein portions of the nonwoven topsheet not having the patterned surfactant are hydrophobic.
3. The absorbent article according to paragraph 1 or 2, wherein the discrete elements are not registered with the second regions, but partially overlap the second regions.
4. The absorbent article according to any one of the preceding paragraphs, wherein the patterned surfactant covers between about 10% and about 60% , preferably between about 10% and about 30%, more preferably between about 10% and about 20%, of a total area of the garment-facing surface of the nonwoven topsheet, according to the Composition Pattern Analysis Test.
5. The absorbent article according to any one of the preceding paragraphs, wherein the absorbent article is a sanitary napkin comprising wings extending outwardly relative to a central longitudinal axis of the sanitary napkin, and wherein the nonwoven topsheet extends into the wings.
6. The absorbent article according to paragraph 5, wherein the garment-facing surface of the nonwoven topsheet present in the wings is free of the patterned surfactant.
7. The absorbent article of paragraph 5, wherein the nonwoven topsheet comprises two or more longitudinally extending barriers inboard of the wings, and wherein the patterned surfactant is positioned between the two or more longitudinally extending barriers.
8. The absorbent article according to any one of the preceding paragraphs, wherein at least some of the plurality of discrete, spaced apart elements form a polygonal shape.
9. The absorbent article according to any one of the preceding paragraphs, wherein the Free Fluid Acquisition Rewet of the absorbent article is about 0.05 grams to about 0.8 grams, preferably about 0.05 grams to about 0.6 grams, or more preferably about 0.05 grams to about 0.55 grams, according to the Acquisition Time and Rewet Test.
10. The absorbent article according to any one of the preceding paragraphs, wherein the Free Fluid Acquisition Time of the absorbent article is about 5 seconds to about 25 seconds, preferably about 5 seconds to about 15 seconds, or more preferably about 5 seconds to about 10 seconds, according to the Acquisition Time and Rewet Test.
11. The absorbent article according to any one of the preceding paragraphs, wherein the first average intensive property and the second average intensive property are basis weight.
12. The absorbent article according to any one of paragraph 1-11, wherein the first average intensive property and the second average intensive property are caliper.
13. The absorbent article according to any one of paragraph 1-11, wherein the first average intensive property and the second average intensive property are volumetric density.
14. The absorbent article according to any one of the preceding paragraphs, wherein the nonwoven topsheet comprises bonds at fiber intersections formed by passing hot air through the nonwoven web.
15. The absorbent article according to any one of paragraphs 1-14, wherein the nonwoven topsheet comprises calendar bonds configured to join the fibers together.
16. The absorbent article according to any one of the preceding paragraphs, wherein the nonwoven topsheet comprises a second, visually discernible pattern of three-dimensional features on the first surface or the second surface, wherein the three-dimensional features comprise one or more third regions and a plurality of fourth regions, wherein the one or more third regions are different than the plurality of fourth regions in a value of an average intensive property, and wherein the second visually discernible pattern of three-dimensional features does not overlap the visually discernible pattern of three-dimensional features.
17. The absorbent article according to any one of the preceding paragraphs, wherein the nonwoven topsheet comprises multicomponent fibers, and wherein at least one component of the multicomponent fibers is bio-based.
18. The absorbent article according to any one of the preceding paragraphs, wherein the nonwoven topsheet has a basis weight in the range of about 10 gsm to about 35 gsm, preferably about 15 gsm to about 30 gsm, or more preferably about 20 gsm to about 30 gsm, according to the Basis Weight Test, and wherein the nonwoven topsheet is a spunbond nonwoven web.
19. The absorbent article according to any one of the preceding paragraphs, wherein a portion of the nonwoven topsheet has a TS7 value in the range of about 1 dB V² rms to about 4.5 dB V² rms, according to the Emtec Test, and wherein the portion of the nonwoven topsheet has a TS750 value in the range of about 6 dB V² rms to about 30 dB V² rms, according to the Emtec Test.
20. The absorbent article according to paragraph 19, wherein the portion of the nonwoven topsheet a D value in the range of about 2 mm/N to about 6 mm/N, according to the Emtec Test.
21. The absorbent article according to any one of the preceding paragraphs, wherein the nonwoven topsheet comprises fibers comprising a hydrophobic melt additive.
22. The absorbent article according to any one of the preceding paragraphs, wherein the nonwoven topsheet comprises continuous fibers, preferably multicomponent continuous fibers.
23. The absorbent article according to any one of the preceding paragraphs, wherein the nonwoven topsheet comprises crimped fibers.
24. The absorbent article according to any one of the preceding paragraphs, wherein the patterned surfactant has a first hydrophilicity, the garment-facing surface of the nonwoven topsheet comprising a continuous surfactant, at least a portion of the patterned surfactant overlapping a portion of the continuous surfactant, the continuous surfactant having a second hydrophilicity that is more hydrophobic than the first hydrophilicity.
25. The absorbent article according to any one of the preceding paragraphs, wherein a ratio of a pattern spacing distance to a pattern width is in the range of about 1.4 to about 5, preferably about 2 to about 3, according to the Composition Pattern Analysis Test.
26. The absorbent article according to any one of the preceding paragraphs, wherein a ratio of a pattern spacing distance to a pattern width is in the range of about 1 to about 8, preferably about 2.5 to about 5.5, according to the Composition Pattern Analysis Test.
27. The absorbent article according to any one of the preceding paragraphs, wherein visually discernable pattern of three-dimensional features has a first pattern, wherein the patterned surfactant has a second pattern, and wherein the first and second patterns are different.
28. The absorbent article according to any one of the preceding paragraphs, wherein the discrete, spaced apart elements are hydrophilic, and wherein portions of the nonwoven topsheet free of overlap with the patterned surfactant are hydrophobic or less hydrophilic than the discrete, spaced apart elements.
29. A nonwoven topsheet comprising:

a first surface;

a second surface; and

a visually discernible pattern of three-dimensional features on the first surface or the second surface, wherein the three-dimensional features comprise one or more first regions and a plurality of second regions;

wherein the one or more first regions have a first value of an average intensive property, wherein the plurality second regions have a second value of the average intensive property, wherein the first value is greater than the second value, and wherein the first value and the second value are greater than zero;

wherein the first regions are continuous;

wherein the second regions are discrete, and wherein at least some of the first regions surround at least some of the second regions;

a patterned surfactant on the first or the second surfaces of the nonwoven topsheet;

wherein the patterned surfactant comprises a plurality of discrete, spaced apart hydrophilic elements;

wherein portions of the nonwoven topsheet not having the patterned surfactant are hydrophobic or less hydrophilic than the hydrophilic elements; and

wherein the discrete, spaced apart elements have an area between about 0.75 mm² and 30 mm², preferably between about 0.75 mm² to about 15 mm², according to the Composition Pattern Analysis Test.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular forms of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this present disclosure.

What is claimed is:

1. An absorbent article comprising:
a nonwoven topsheet;
a liquid impermeable backsheet;
an absorbent core positioned at least partially intermediate the topsheet and the backsheet;
the nonwoven topsheet comprising:
a first surface;
a second surface; and
a visually discernible pattern of three-dimensional features on the first surface or the second surface, wherein the three-dimensional features comprise one or more first regions and a plurality of second regions;
wherein the one or more first regions have a first value of an average intensive property, wherein the plurality second regions have a second value of the average intensive property, wherein the first value is greater than the second value, and wherein the first value and the second value are greater than zero;
wherein the first regions are continuous;
wherein the second regions are discrete, and wherein at least some of the first regions surround at least some of the second regions;
a patterned surfactant on a garment-facing surface of the nonwoven topsheet;
wherein the patterned surfactant comprises a plurality of discrete, spaced apart elements; and
wherein the discrete, spaced apart elements have an area between about 0.75 mm² and 30 mm², according to the Composition Pattern Analysis Test.

2. The absorbent article according to claim 1, wherein portions of the nonwoven topsheet not having the patterned surfactant are hydrophobic.

3. The absorbent article according to claim 1, wherein the discrete elements are not registered with the second regions, but partially overlap the second regions.

4. The absorbent article according to claim 1, wherein the patterned surfactant covers between about 10% and about 30% of a total area of the garment-facing surface of the nonwoven topsheet, according to the Composition Pattern Analysis Test.

5. The absorbent article according to claim 1, wherein the absorbent article is a sanitary napkin comprising wings extending outwardly relative to a central longitudinal axis of the sanitary napkin, and wherein the nonwoven topsheet extends into the wings.

6. The absorbent article according to claim 5, wherein the garment-facing surface of the nonwoven topsheet present in the wings is free of the patterned surfactant.

7. The absorbent article of claim 5, wherein the nonwoven topsheet comprises two or more longitudinally extending barriers inboard of the wings, and wherein the patterned surfactant is positioned between the two or more longitudinally extending barriers.

8. The absorbent article according to claim 1, wherein at least some of the plurality of discrete, spaced apart elements form a polygonal shape.

9. The absorbent article according to claim 1, wherein the Free Fluid Acquisition Rewet of the absorbent article is about 0.05 grams to about 0.6 grams, according to the Acquisition Time and Rewet Test, and wherein the Free Fluid Acquisition Time of the absorbent article is about 5 seconds to about 15 seconds, according to the Acquisition Time and Rewet Test.

10. The absorbent article according claim 1, wherein the first average intensive property and the second average intensive property are basis weight, caliper, or volumetric density.

11. The absorbent article according to claim 1, wherein the nonwoven topsheet comprises bonds at fiber intersections formed by passing hot air through the nonwoven web, or wherein the nonwoven topsheet comprises calendar bonds configured to join the fibers together.

12. The absorbent article according to claim 1, wherein the nonwoven topsheet comprises a second, visually discernible pattern of three-dimensional features on the first surface or the second surface, wherein the three-dimensional features comprise one or more third regions and a plurality of fourth regions, wherein the one or more third regions are different than the plurality of fourth regions in a value of an average intensive property, and wherein the second visually discernible pattern of three-dimensional features does not overlap the visually discernible pattern of three-dimensional features.

13. The absorbent article according to claim 1, wherein the nonwoven topsheet comprises continuous multicomponent fibers, and wherein at least one component of the multicomponent fibers is bio-based.

14. The absorbent article according to claim 1, wherein a portion of the nonwoven topsheet has a TS7 value in the range of about 1 dB V² rms to about 4.5 dB V² rms, according to the Emtec Test, and wherein the portion of the nonwoven topsheet has a TS750 value in the range of about 6 dB V² rms to about 30 dB V² rms, according to the Emtec Test.

15. The absorbent article according to claim 1, wherein the nonwoven topsheet comprises fibers comprising a hydrophobic melt additive.

16. The absorbent article according to claim 1, wherein the nonwoven topsheet comprises continuous fibers.

17. The absorbent article according to claim 1, wherein the patterned surfactant has a first hydrophilicity, the garment-facing surface of the nonwoven topsheet comprising a continuous surfactant, at least a portion of the patterned surfactant overlapping a portion of the continuous surfactant, the continuous surfactant having a second hydrophilicity that is more hydrophobic than the first hydrophilicity.

18. The absorbent article according to claim 1, wherein a ratio of a pattern spacing distance to a pattern width is in the range of about 1 to about 8, according to the Composition Pattern Analysis Test.

19. The absorbent article according to claim 1, wherein visually discernable pattern of three-dimensional features has a first pattern, wherein the patterned surfactant has a second pattern, and wherein the first and second patterns are different.

20. The absorbent article according to claim 1, wherein the discrete, spaced apart elements are hydrophilic, and wherein portions of the nonwoven topsheet free of overlap with the patterned surfactant are hydrophobic or less hydrophilic than the discrete, spaced apart elements.

21. A nonwoven topsheet comprising:
a first surface;
a second surface; and
a visually discernible pattern of three-dimensional features on the first surface or the second surface, wherein the three-dimensional features comprise one or more first regions and a plurality of second regions;
wherein the one or more first regions have a first value of an average intensive property, wherein the plurality second regions have a second value of the average intensive property, wherein the first value is greater than the second value, and wherein the first value and the second value are greater than zero;
wherein the first regions are continuous;
wherein the second regions are discrete, and wherein at least some of the first regions surround at least some of the second regions;
a patterned surfactant on the first or the second surfaces of the nonwoven topsheet;
wherein the patterned surfactant comprises a plurality of discrete, spaced apart hydrophilic elements;
wherein portions of the nonwoven topsheet not having the patterned surfactant are hydrophobic or less hydrophilic than the hydrophilic elements; and
wherein the discrete, spaced apart elements have an area between about 0.75 mm² and 30 mm², preferably between about 0.75 mm² to about 15 mm², according to the Composition Pattern Analysis Test.
